(19) United States
(12) Patent Application Publication
(10) Pub. No.: US 2023/0114401 A1
(43) Pub. Date: Apr. 13, 2023

(54) METHOD AND DISINFECTION SYSTEM FOR DISINFECTING A CLOSED SPACE OR SURFACE INCLUDING WALL-MOUNTED UV RADIATION DELIVERY AND MONITORING APPARATUS

(71) Applicant: STERILIZ, LLC, Rochester, NY (US)

(72) Inventor: Samuel Richard Trapani, Rochester, NY (US)

(21) Appl. No.: 18/081,620

(22) Filed: Dec. 14, 2022

Related U.S. Application Data
(63) Continuation-in-part of application No. 17/244,433, filed on Apr. 29, 2021.
Application No. 17/244,433 is a continuation-in-part of application No. 16/164,150, filed on Oct. 18, 2018, now Pat. No. 11,154,629.
Application No. 16/164,150 is a continuation-in-part of application No. 15/169,164, filed on May 31, 2016, now Pat. No. 10,226,541.
Application No. 15/169,164 is a continuation of application No. 14/263,774, filed on Apr. 28, 2014, now Pat. No. 10,010,633.
Application No. 14/263,774 is a continuation-in-part of application No. 13/964,874, filed on Aug. 12, 2013, now Pat. No. 9,345,798.
Application No. 13/964,874 is a continuation-in-part of application No. 13/446,563, filed on Apr. 13, 2012, now abandoned.
(60) Provisional application No. 63/029,292, filed on May 22, 2020.
(60) Provisional application No. 61/475,722, filed on Apr. 15, 2011.
(60) Provisional application No. 63/322,156, filed on Mar. 21, 2022.

(51) Int. Cl. A61L 2/10; A61L 2/24; A61L 2/20; A61L 9/015; A61L 9/20

(52) U.S. Cl. CPC A61L 2/10; A61L 2/24; A61L 2/202; A61L 2/208; A61L 9/015; A61L 9/20; G01J 1/429

(57) ABSTRACT

Method and disinfection system for disinfecting a closed space or surface including light fixtures fixedly positioned at predefined locations in a closed space. The disinfection system includes a UV emitter module positioned over a robotic system. The light fixtures and the UV emitter module emit ultraviolet C (UVC) light for disinfecting the closed space or a surface in the closed space. The disinfection system includes sensors for measuring the amount of UVC light delivered by the light sources and UV emitter module. The disinfection system includes a control system for determining a profile for the closed space or surface based on the amount of UVC light needed and corresponding time to disinfect. The control system communicatively connects to the sensors. The control system monitors the amount of UVC light and controls the operation of the light sources and UV emitter module to deliver the required amount of UVC light.

### CROSS-REFERENCE TO RELATED APPLICATIONS

This application claims priority to U.S. Provisional Application 63/322,156, titled “Fixed and Stationary Room Ultraviolet C Radiation Disinfection System and A Method Of Disinfecting An Enclosed Space Or Surface,” having Attorney Docket No. STRL006USP1 and filed Mar. 21, 2022; and

U.S. Non-Provisional patent application Ser. No. 17/244,433, titled “Method and Sterilization System For Improving Duty Cycle Of Robotic System And Ultraviolet (UV) Emitters Disinfecting Closed Environment,” having Attorney Docket No. STRL001US5, which is pending and claims priority to a U.S. Provisional Patent Application No. 63/029,292, filed May 22, 2020;

which is a continuation-in-part of U.S. application Ser. No. 16/164,150, filed Oct. 18, 2018 (now U.S. Pat. No. 11,154,629);

which is a continuation-in-part of U.S. application Ser. No. 15/169,164, filed May 31, 2016 (now U.S. Pat. No. 10,226,541);

which is a continuation of U.S. application Ser. No. 14/263,774, filed Apr. 28, 2014 (now U.S. Pat. No. 10,010,633);

which is a continuation-in-part of U.S. application Ser. No. 13/964,874, filed Aug. 12, 2013 (now U.S. Pat. No. 9,345,798);

which is a CIP of Ser. No. 13/446,563, filed Apr. 13, 2012 (now abandoned); which claims priority to U.S. Provisional Patent Application No. 61/475,722, filed Apr. 15, 2011; and

U.S. application Ser. No. 15/169,164, filed May 31, 2016 (now U.S. Pat. No. 10,226,541) is a divisional of U.S. application Ser. No. 13/964,874, filed Aug. 12, 2013 (now U.S. Pat. No. 9,345,798);

And all of above-listed patent applications are hereby expressly incorporated by reference in their entirety.

### FIELD OF THE INVENTION

The present invention relates to disinfection. More specifically, the present invention relates to disinfection of pathogens using fixed light fixtures and, and a UV emitter module positioned over a robotic system that is configured to maneuver the UV emitter module.

### BACKGROUND OF THE INVENTION

In the U.S. each year, more people die from hospital infections than from AIDS, breast cancer, and auto accidents combined. These infections are the fourth leading cause of death, with current annual estimates of 2,000,000 infections, 100,000 deaths, and added costs of $45 billion. Somewhere between 5-10% of all patients admitted to a hospital acquire an infection while in that hospital. Even with such extreme statistics, many industry experts consider the problem to be severely underestimated.

Currently, Clostridium difficile (C. diff) is one of the most problematic pathogens in the healthcare industry. It causes C. diff Infection (CDI) that sickens and kills humans. The Centers for Disease Control (CDC) states that C. diff spores are transferred to patients mainly via the hands of healthcare personnel who have touched a contaminated surface or item. They also say that to reduce the chance of spreading pathogens and subsequent disease, surfaces must be cleaned better.

Current cleaning methods such as a chemical disinfection are not working as a stand-alone practice. Additional measures need to be taken to reduce patient and healthcare professionals' risks. UV light is known to destroy pathogens in water supplies and HVAC systems. In recent years, products also have been developed that utilize UV to disinfect surfaces and air.

It is well-known that the UV portion of the light spectrum has the ability to inactivate the DNA of pathogens, making them unable to multiply. Medical uses of UV light include sterilization of surfaces and air without the use of chemicals. Well documented studies identify the specific amount (dosage) of UV light necessary to disinfect and sterilize surfaces and air.

One type of existing UV disinfection equipment uses either a manually operated switch or a timer to deliver UV light for a period of time. Another type of existing UV disinfection equipment is a system that measures reflected UV light, generally at the emitter, to control how much time the UV light is delivered. Both of these types of equipment are unable to definitively deliver a specific dosage to a surface or volume of air because no measurement is taken at the location of interest. This problem results in surfaces and air being over- or under-treated. In the case of over-treating, excess treatment time slows down the facility's operations, thus adding to operating costs and reducing throughput, and results in excess exposure to room surfaces, which causes faster breakdown of the materials from which the surfaces are made. In the case of under-treating, disinfection or sterilization is not assured, resulting in reduced efficacy and increased exposure to liability lawsuits.

In addition, UV disinfection equipment is placed over a base. Subsequently, the base is maneuvered either manually or automatically to a desired area to sterilize or disinfect desired area. Generally, a user has to be present at all times to maneuver the UV disinfection equipment to the desired area for operating the UV disinfection equipment. Alternatively, UV disinfection equipment mounts to a motorized base or robotic system permanently or semi-permanently. The motorized base or robotic system includes a power source such as a battery for maneuvering the robotic UV disinfection equipment. Since it takes significant time to recharge the battery, the robotic UV disinfection equipment cannot be used for disinfecting desired area when it is being charged thus resulting in reduced duty cycle for the robotic UV disinfection equipment.

Additionally, several systems for disinfecting closed spaces and/or surfaces to inactive viruses have been disclosed in the past. Some of the disinfecting systems deliver UVC light onto the surfaces to disinfect and sterilize the surfaces. One such example is disclosed in a U.S. granted Pat. No. 9,956,306, entitled, “Room decontamination system, method and controller” (“the '306 Patent”). The '306 Patent discloses room decontamination systems, controllers and methods for decontaminating a closed space or surface. The room decontamination system may be a UV room decontamination system that uses UV radiation to perform a decontamination operation in the room. A controller may determine whether safe conditions for decontamination exist and initiate a decontamination operation on the basis of whether they exist. Determination of safe conditions for decontamination may be based on light actuation detection and/or sensor data, which may include presence detector data and door sensor data. Determination of safe conditions for decontamination may include a determination of whether sensors are functioning properly. The controller may also determine whether decontamination operations are required on the basis of the historical condition data, for example on the basis of whether the room has been occupied since the last decontamination operation.

Another example is disclosed in a U.S. granted Pat. No. 10,556,025, entitled “Fixed position hybrid germicidal irradiation apparatus, method, and system” (“the '025 Patent”). The '025 Patent discloses a fixed position hybrid germicidal irradiation apparatus, method, and system for ultraviolet germicidal irradiation. A plurality of emitters may be coupled to a substantially rectangular housing configured to be coupled to a standard commercial lighting fitting. A second plurality of emitters may be coupled to the substantially rectangular housing. The first plurality of emitters and the second plurality of emitters are operable to emit UV-C radiation at a wavelength of about 265 nanometers and near-UV radiation at a wavelength of about 405 nanometers respectively. UV-C sensors are configured to measure the amount of UV-C light or near UV-C light from a target surface. A controller may be configured to engage with the UV-C sensors to determine the amount of UV-C radiation collected by the UV-C sensors. The improved apparatus, method, and system reduces exposure time by varying the intensity and wavelength of the UV administered, while concurrently reducing UV overexposure.

Another example is disclosed in a U.S. granted Pat. No. 9,345,798, entitled “Room sterilization method and system” (“the '798 Patent”). The '798 Patent discloses a sterilization system consisting of a mobile emitter, a sensing subsystem and a data logging subsystem is described. The emitter has one or more UV emitting lamps or devices. The sensing system comprises at least one remote UV sensor and at least one door sensor. The door sensor comprises a safety shut off door detector and may contain an emergency stop detector and arming detector to protect people from being exposed to UV energy. The system has a remote control for starting, stopping and setting system parameters which include but are not limited to: treatment time, dosage, room size, room number, unit number, floor, facility name, operator name, operator identification number, password, default dosage values, dosage, and patient identification number. The number of treatments per unit of time can be maximized because of the use of incident light measurement.

Although the above-discussed systems provide effective ways to disinfect closed space or surface, they have few problems. For example, the UV light emitters are not placed properly and as such the UV light is not delivered properly onto the required surface. This increases the time required to disinfect targeted areas in the closed space or surface.

Therefore, there is a need in the art to provide a room disinfection system having light fixtures permanently mounted to the perimeter walls or ceiling or other surface of the closed space, the light fixtures having UV light source to deliver the required UV dose for disinfecting targeted areas in the closed space or surface.

### SUMMARY OF THE INVENTION

The present invention provides room disinfection and/or sterilization methods and apparatuses that satisfy the demands discussed above while improving on known shortcomings in the conventional art.

The present invention includes a mobile system which, when used properly, automatically delivers UV-C light to all surfaces within a treatment space, including hard-to-clean, high-touch surfaces such as keyboards, computers, and bed rails, yielding a significantly cleaner, safer environment.

When compared to other systems and techniques, the present invention provides faster processing-time and critical data capture that clearly demonstrates treated areas have received the necessary dosage of UV-C light needed to eradicate dangerous pathogens effectively. Another benefit of UV-C disinfection is that it leaves no harmful residue or vapors. The room is immediately available for occupancy.

The present invention includes a room disinfection and sterilization system utilizing UV light to cause permanent and fatal injury to the pathogens that cause illness and death to humans. This system includes an emitter that produces UV light, one or more remote wired or wireless UV light sensors that measure the intensity of UV light incident upon them, one or more remote wired or wireless door sensor safety stop switches which cause the system to stop emitting UV light in the event the door being monitored is opened or if the safety stop switch is depressed, a wired or wireless remote control to operate the system, and a central computer with wireless local area network (WLAN, e.g., Wi-Fi, WICAD, Bluetooth, 802.11, 802.15, 3G, 4G, CDMA, or other suitable technology) connect-ability to access the internet and provide local and remote logging of disinfection cycles or jobs. In some embodiments, the device of the present disclosure may include its own Wi-Fi access point, along with an Internet bridge (for example, a cellular modem). In some embodiments, the remote control may be a standard iPod or other PDA/smartphone/tablet/handheld device.

The present invention delivers lethal UVC light doses into all areas of complex environments, destroying viruses, spores, and drug-resistant bacteria, even in a room's shadowed areas. Its unique, next-generation remote sensors make this the only system that can measure how much UVC energy reaches every corner of a treatment space.

The present invention employs at least one remote sensor which definitively measures the precise dosage of UV light delivered to the location of interest. Data recording and reporting are necessary for tracking and analyzing infections acquired in facilities where disinfection occurs. A significant difference between the present invention and conventional equipment and systems is the ability to store, retrieve, report and analyze such data. The present invention employs local storage of such data on the hard disk drive of its computer controller and remote data storage via up-linking to a remote internet host server for others to access.

Studies have shown that a significant problem with UVC disinfection of surfaces occurs when the subject surface is not exposed to direct UVC light. Instead, the subject surface may be exposed to UVC light that is reflected off other surfaces. The intensity of this reflected light is often much lower than that of direct UVC light resulting in lower inactivation of pathogens. One possible solution to help improve inactivation of pathogens is to reposition the UVC emitter throughout the disinfection treatment of the space being treated. Another possible solution is to use multiple UVC emitters. In one preferred embodiment of the present invention, a multiple emitter configuration is used to disinfect the space. An issue with using multiple emitters is the distance between the control station and the emitters. The usable distance for the control station to communicate, in an indoor environment such as a hospital, with an emitter, is limited to approximately 30 feet. The present invention solves the distance limitation by establishing data communication between the control station and only one emitter, which is a Master emitter, and which remains located within the limited distance. The other emitters located within the space to be disinfected are Slave emitters, which communicate with the Master emitter rather than the control station. Another issue with multiple emitters within a given space is a limited amount of AC power available in the space. A typical UVC emitter draws 15 amps. If more than one emitter is plugged into the same AC outlet, and are energized at the same time, the resulting electrical overload may trip a circuit breaker. resulting in a loss of power to two emitters during the disinfection process. The present invention solves this problem by monitoring either voltage drop or current draw, and shutting off the emitter or emitters using too much power.

In addition, the present invention provides a sterilization system including separate Ultraviolet (UV) emitter modules and robotic system, each having their own resources such that robotic system may return UV emitter module with discharged battery to a charging dock and leave it there to charge and pick up another UV emitter module with fully charged battery to disinfect the closed environment.

The sterilization system includes a robotic system and a plurality of Ultraviolet (UV) emitter modules. The robotic system receives a signal corresponding to discharge of battery from one of plurality of UV emitter modules. Each of the plurality of UV emitter modules includes a Ultraviolet (UV) light source for disinfecting the closed environment. The robotic system detects location of the UV emitter module amongst the plurality of UV emitter modules and maneuvers to location of the UV emitter module.

After reaching the location of the UV emitter module, robotic system mounts the UV emitter module and maneuvers the UV emitter module to a charging dock for charging a battery of the UV emitter module. After charging, the robotic system maneuvers the UV emitter module to a predefined area for disinfecting predefined area of the closed environment. In other words, the robotic system maneuvers the UV emitter module with discharged battery to the charging dock and leaves it there to charge. After charging the battery, the robotic system picks up the UV emitter module with the battery fully charged to disinfect the closed environment.

In one embodiment, the robotic system includes an emitter interface for connecting the robotic system to the UV emitter module. The emitter interface allows charging the battery of the robotic system when the charging dock charges the battery of UV emitter module. Alternatively, the emitter interface allows charging battery of the robotic system through the UV emitter module when the UV emitter module includes a battery that is fully charged.

In one advantageous feature of present invention, the battery of the robotic system may be continuously get charged or recharged from connected the UV emitter modules, thereby allowing robotic system and UV emitter modules to have a 100% duty cycle. This ensures uninterrupted operation of the robotic system and the UV emitter modules for disinfecting the closed environment using the UV emitter modules. Further, this saves cost and man hours for users.

In addition, the present invention discloses a room disinfection system for disinfecting a closed space or surface. The room disinfection system includes a plurality of light fixtures (two or more light fixtures) fixedly positioned at predefined locations such as walls or ceiling in a closed space such as a room. Each of the plurality of light fixtures comprises a light source that emits ultraviolet C (UVC) light for disinfecting the closed space or surface. In one example, each light source emits ultraviolet C (UVC) radiation with wavelengths between 200 and 290 nanometer (nm). The UVC light kills microorganisms by damaging the genetic material, and the deoxyribonucleic acid (DNA) of the microorganisms.

The room disinfection system includes a plurality of sensors for measuring the amount of UVC light delivered by the light source in each of the plurality of light fixtures. The room disinfection system includes a control system communicatively connecting the plurality of sensors. The control system receives the amount of UVC light delivered by the light source in each of the plurality of light fixtures from the plurality of sensors. Further, the control system controls the operation of the light sources to deliver the required amount of UVC light.

In one embodiment, the room disinfection system includes one or more sensors. The room disinfection system utilizes the data received from the one or more sensors periodically to record a profile based on the dose delivered. This is made possible as some rooms have furniture, fixtures and equipment locations that do not change. In the present embodiment, the amount of time needed to deliver a desired dose is determined by the control system by looking up the dose delivered profile and finding the corresponding time to deliver the desired dose. This technique allows changing doses without having to recalibrate each time a dose is changed.

The room disinfection system delivers an ultra-fast, and effective dosage of UVC light for killing harmful pathogens when disinfecting closed spaces (rooms), or any other surface without having to be moved or positioned with each use. For example, the room disinfection system helps to disinfect closed spaces or surfaces between each patient case in as little as two (a) minutes depending on the size of the closed space.

A further feature includes the ability to run several calibration cycles with one or more sensors with the sensors placed in different areas for each cycle. Upon completion of the several calibration cycles the control system then uses the profile for the cycle with the longest time, or a profile based upon some other algorithm, thereby delivering at least the target dosage to all areas where the sensors were placed for the calibration cycles.

In another implementation, the disinfection system includes light fixtures fixedly positioned at predefined locations in a closed space. The disinfection system includes a UV emitter module positioned over a robotic system. The light fixtures and the UV emitter module emit ultraviolet C (UVC) light for disinfecting the closed space or a surface in the closed space. The disinfection system includes sensors for measuring the amount of UVC light delivered by the light sources and the UV emitter module. The disinfection system includes a control system for determining a profile for the closed space or the surface based on the amount of UVC light needed and corresponding time to disinfect. The control system communicatively connects to the sensors. The control system monitors the amount of UVC light and controls the operation of the light sources and the UV emitter module to deliver the required amount of UVC light.

In one advantageous feature of the present invention, the room disinfection system comprises the light fixtures mounted at specific locations such as walls or ceiling. This fixed location of the light fixtures helps to direct the UVC light from the light sources and quickly deliver to target life-threatening, health care-associated infection (HAI) pathogens and disinfect an entire closed space with higher efficacy. This reduces the time required to sanitize or disinfect the closed space or surface such as a dental chair in the closed space.

In one advantageous feature of the present invention, the sensors measure the dosage of UVC light delivered from the light sources. The control system monitors whether the lamps are ON and functioning properly using the sensors based on the profile of the space that needs to be sanitized. The sensors measure and communicate it to the control system such that the required UVC light (UVC light equal to or greater than required amount) is delivered to the targeted areas in the closed space. In one example, the control system captures critical information like date, time, cycle time and the like.

In another advantageous feature of the present invention, the light fixtures include highly polished, precision tuned reflectors to maximize the dosage of UVC light and minimize treatment time, thereby keeping the closed space available for use most of the time. The reflectors have a special geometric feature that minimizes, or eliminates, UVC light emitted from the lamp to be reflected back to the lamp. When UVC light is emitted then reflected back to the lamp a decrease in the amount of UVC delivered to the room is realized.

In another advantageous feature of the present invention, the control system includes a display having a touch control panel. A user operates the touch control panel to control the operation of the light sources through the control system. Further, the display displays the status of operation of the light sources such that no one enters the closed space when the light sources are ON and disinfecting the closed space or surface.

These and other advantages of the present invention will be apparent from the description provided herein. Additional systems, methods, features and advantages of the present invention will become apparent to one with ordinary skill in the art upon examination of the following drawings and detailed description of the preferred embodiments.

### BRIEF DESCRIPTIONS OF THE DRAWINGS

The features, nature, and advantages of the present invention will become more apparent from the detailed description set forth below in conjunction with the drawings, wherein:

FIG. 1 is a perspective view of a disinfection system, in accordance with the present invention;

FIG. 2 is a process flow of a method of using the disinfection system, in accordance with the present invention;

FIGS. 3-12 are screenshots of a remote operating device, in accordance with the present invention;

FIG. 13 is a perspective view of a preferred embodiment of the present invention, including a Master UV emitter transmitting signals to three Slave emitters;

FIG. 14 is a perspective view of a preferred embodiment of the present invention, similar to the embodiment of FIG. 13, including a Master UV transmitting a signal to a first slave UV emitter, which is rebroadcasting the signal to a second slave UV emitter out of range of the Master UV emitter;

FIG. 15 illustrates an environment in which sterilization system is implemented, in accordance with one embodiment of present invention;

FIGS. 16 through 19 illustrate block diagrams of Ultraviolet (UV) emitter module, robotic system, server and user device, respectively;

FIGS. 20 through 23 illustrate operational features of robotic system and UV emitter modules allowing 100% duty cycle of robotic system and UV emitter modules;

FIG. 24 illustrates a method implemented by robotic system, in accordance with one embodiment of present invention;

FIGS. 25A and 25B illustrate an environment in which a room disinfection system installs to disinfect a closed space or surface, in accordance with one embodiment of the present subject matter;

FIG. 26 illustrates a feature of a light fixture;

FIG. 27 illustrates a block diagram of the room disinfection system;

FIG. 28 illustrates a display installed at the door;

FIGS. 29 and 30 illustrate a reflector having different geometric features for minimizing or eliminating UVC light emitted from the light fixture to be reflected back to the light fixture, in in accordance with one embodiment of the subject matter;

FIG. 31 illustrates a method of disinfecting a closed space or surface, in accordance with one embodiment of the subject matter; and

FIG. 32 illustrates a method of disinfecting a closed space or surface by calibrating a control system based on the profile of the closed space or surface, in accordance with one embodiment of the subject matter.

### DETAILED DESCRIPTION OF THE PREFERRED EMBODIMENTS

The present invention will be understood more readily by reference to the following detailed description of the presently preferred embodiments.

Reference will now be made in detail to certain presently preferred embodiments of the invention, examples of which are illustrated in the accompanying drawings. Wherever possible, the same reference numerals are used throughout the drawings to refer to the same or like parts.

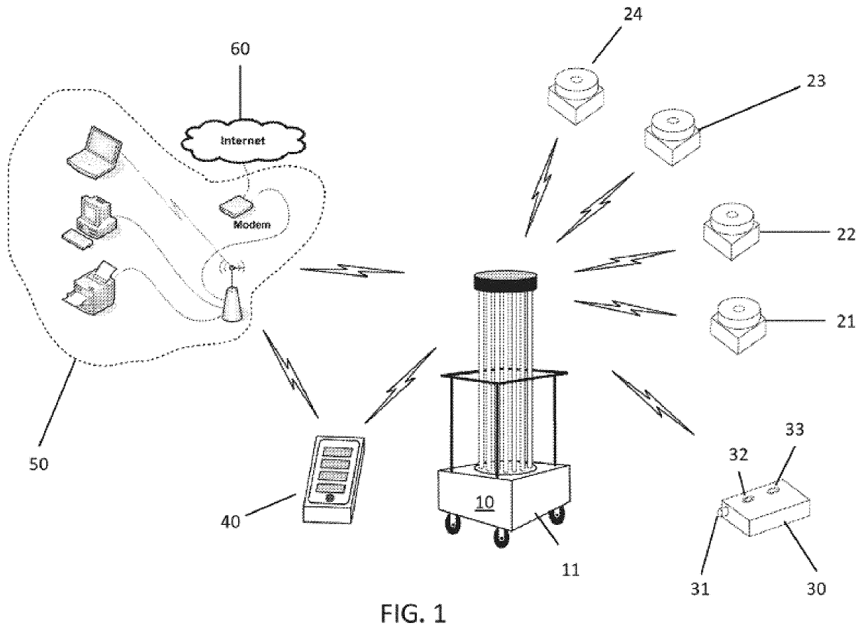

As broadly embodied in FIG. 1, a sterilization system includes an emitter 10, a sensing system consisting of UV sensors 21-24, door sensor 30, a remote control 40, an optional wireless local area network (WLAN) 50, and an optional Internet connection 60 for the optional WLAN.

In more detail with reference to FIG. 1, the emitter subsystem includes the emitter 10 which is a mobile structure on wheels with a plurality of lamps that emit ultraviolet light when energized by their corresponding electronic ballast(s). Other possible embodiments of an emitter may be a mobile structure with another means for transporting without the use of integrated wheels, or a stationary structure. This subsystem further includes a central computer 11, which executes a software program that controls energization of the UV lamps and ballasts through the cycling of an interconnected relay. Computer 11 communicates with the sensing subsystem's remote UV sensors 21-24, which provide UV light level readings used by computer 11 to determine when a previously programmed dose of incident UV light has been delivered to each of the remote sensor locations.

The dosage setting is determined by the specific pathogen(s) the operator wants to eradicate. For example, to deliver a disinfection dosage of UV light for the elimination of Escherichia coli (E. coli) requires 6,600 microwatt-seconds/centimeter-squared for a 100:1 reduction (2 log) in E. coli; thus, the setting would be 6,600. Existing systems and equipment do not utilize remote sensor(s) for surface and air disinfection therefore they do not offer a way to definitively deliver this dosage to a remote location. The appropriate dosages to provide a particular log reduction in a particular pathogen are well known in the art. The present invention is not limited to any particular pathogen, disinfection level, or UV dosage.

As UV light is harmful to humans, computer 11 communicates with door sensor 30, and if the monitored door has been opened during a disinfection cycle, which may indicate a person has entered the room being treated, the lamps are immediately turned off to prevent injury. The human-computer interface (HCl) may be accomplished via remote control 40, which is used to configure the type and number of sensors to be used for a particular job; to select the minimum dosage of UV light to deliver to each UV sensor; to begin, pause and end a job; and to view job reports stored on computer 11. Pausing the job may be advantageous for various reasons, including the possibility of re-positioning the emitter mid-job in order to obtain fuller coverage of the room. Computer 11 communicates with a remote web server, via the optional internet connected WLAN 50 (if present), to post job reports and status for remote access by password-protected users. The emitter subsystem contains a battery that keeps the computer energized when the subsystem is not plugged into the facility's electric utility. This battery is automatically recharged as needed from the facility's electric when the subsystem is plugged in. The emitter subsystem also has docks for recharging system sensors that operate by battery power.

Optional components of the present invention may be included in order to increase efficacy against particular pathogens. For example, in addition to using UV light, the device may further use a humidity generating device, an ozone generating device, and/or a vaporized hydrogen peroxide gas generating device. The combination of these optional components may provide better and faster disinfection than UV alone.

An integral part of the sterilization system is the sensing subsystem of FIG. 1. The UV light sensing system will measure actual incident light at least one particular site(s) in an area. An advantage of using incident light over reflected light is that with incident light, sensors can be placed at any site to guarantee that UV radiation reaches a certain minimum dosage at that site. UV sensors 21-24 are designed to measure the total UV light incident upon them. Once all active sensors have received the desired dose, the disinfection procedure is considered complete. One method of measuring total incident UV light is to employ a cosine-corrected sensor, which accounts for all light incident upon it from a full 180-degree solid angle. Another method utilizes multiple sensors, incorporated into a single sensor array, to measure the UV light from various angles of incidence and then to utilize an algorithm to integrate the total light incident upon the sensors to obtain the total incident light from a full 180-degree solid angle. Other known methods exist to measure the total incident UV light upon a sensor, and they are not precluded. For example, the sensors may make one or more instantaneous light measurements (perhaps after waiting for an initial warm-up period), and then calculate total dosage by assuming that the light level remains constant. Unless all the energy is integrated accurately, regardless of the method, it is difficult to determine the actual dosage of UV light delivered. Further, by measuring incident light, the system will not over-radiate an area. This allows for the shortest time for disinfection treatment before re-deployment into another area.

Over-radiating is disadvantageous in the fact that that the rays could cause damage to surfaces in the area unnecessarily. This is in addition to the fact that fewer areas can be finished per unit time. When operating, the UV sensor(s) are designed and programmed to continually measure incident UV light. The intensity measured is converted into a voltage, which is sampled by an analog to digital (A/D) converter, which is part of the UV sensor sub-system. The digital data may then be communicated to central computer 11. In addition to the intensity, a remote sensor may communicate a value representing the charge state of its internal battery. In this way, central computer 11 can be informed ahead of time how much battery life may be left in each remote sensor. The preferred embodiment of this communication system is a wireless system, but a wired communication system also is within the scope of the invention. Wireless operation can be accomplished from any number of remote UV sensors back to central computer 11 using one of any number of wireless protocols. A preferred implementation uses four UV sensors 21-24, but any number of UV sensors is within the scope of the invention. A preferred wireless protocol is the 802.15.4 Zigbee standard. In addition to wireless UV sensors 21-24, wireless door sensor 30 is included in the network of FIG. 1.

Wireless UV sensors 21-24 preferably are rechargeable battery-operated units although non-rechargeable or AC-powered units are not precluded. While in operation, the unit may operate completely on battery power. When not in operation, the sensor can be stored in its cradle on the emitter unit, or some other location, at which time re-charging occurs. The set of sensors are operated such that they transmit with different time periods allowing for a lower probability that two units will transmit at the same time. Another preferred embodiment uses buffering of the data at central computer 11 so that if multiple transmissions by sensors occur, the receiver at central computer 11 will not miss any one of them. In yet another preferred embodiment, a central computer 11 initiates the request for data from the door and the UV sensors on a one-by-one basis thereby eliminating the potential for two units to transmit at the same time. The UV sensors 21-24 are expected to see a relatively constant incident light intensity, and as such, time between transmissions, or the transmission period, can be relatively long. An example transmission period is 10 seconds, but this could range from as short as a few milliseconds to as long as several hours and is easily changed by the operator via the system software.

In the case of a nominal 10 second transmission period, the individual sensors can in one embodiment be programmed to retransmit at a time period equal to a prime number of milliseconds close to 10 seconds or 10,000 milliseconds. For example, sensor 1 might transmit at 9949 milliseconds, sensor 2 at 9967 milliseconds, sensor 3 at 9973 milliseconds and sensor 4 at 10007 milliseconds. These are all prime numbers of milliseconds close to 10 seconds. Setting the transmission period to these time periods greatly reduces the probability of two or more transmissions occurring at the same time. This does not preclude the use of similar time periods amongst UV sensors 21-24 as well.

Central computer 11 may receive the UV sensors' transmissions at the programmed transmission times and then calculate the time difference between receptions of information from each individual sensor in order to integrate the total incident light energy from each sensor and ensure that at least the prescribed dosage has been delivered to each particular sensor location.

This scheme provides another safeguard, because central computer 11 can detect if no messages are being received from a particular sensor for some maximum time from the time the last transmission was received. This condition could occur because of a completely discharged battery or a defect in the sensing unit, in which case operator interaction would be required.

Each system may be equipped with door sensor 30 (or in some embodiments multiple door sensors) for emergency shutoff purposes. Door sensor 30 includes door detector 31. If the door opens or closes, central computer 11 will receive a notification from door detector 31. If this event occurs while emitter 10 is operating and emitting UV light, then central computer 11 will shut down emitter 10. In addition to door detector 31, door sensor 30 also includes emergency shutoff detector 32, which could be used by an operator to shut down the system even if the door is closed. All of these events are distinguishable by central computer 11 by the message sent to it from door sensor 30. Central computer 11 knows which of the above-mentioned events occurred because of the digital information transmitted to it.

Latching may be provided on emergency shutoff detector 32. Once emergency shutoff detector 32 is actuated, the indication will remain until reset. The current reset condition occurs when the door is open; however, other methods to reset are possible. Door sensor 30 may also communicate its battery state back to central computer 11 so that the system will know when the battery needs to be recharged, and the operator will be notified.

Once the remote sensor(s) and UV emitter are placed where desired, the operator exits the room and begins the activation sequence by placing door sensor 30 in position at the door such that the sensor can detect if the door is opened or closed, or if there is a change from open to closed or vice-versa.

Next, the operator must actuate arming detector 33 located on the door sensor 30. A designed safety feature prevents the system from being armed unless door sensor 30 indicates the door is closed. An open door or a door that is closed when armed and then opened will reset the system to the unarmed state. This design requires the operator to make sure that the door is closed before re-arming emitter 10.

This is of course only one embodiment of a method to arm the system. Another wired or wireless arming detector placed directly on the emitter could also provide a similar function. Under this condition, the door must be open to arm the system. If the door is open, the operator can actuate the arming detector 33 on the emitter 10. After this detector 33 is actuated and latched (programmed to keep its state as “armed”), the door must close. Once the door is closed, remote control 40 could start the system. If the door has not closed, remote control 40 will not be able to start emitter 10. If the door opened after the emitter 10 started, the emitter 10 would shut down and cease emitting UV light.

Emitter 10 may include one or more additional “drone” emitters. These are additional lights that may be added to the space in order to increase coverage and/or brightness and thereby decrease cleaning time. The drone emitter(s) may be controlled by central computer 11 either by wired or wireless communications.

Door sensor 30 may encode the different switch conditions into a unique voltage measurement. This voltage may be converted to a digital value by an A/D converter, which may be part of door sensor 30 and then sent to central computer 11. The existence of the 3 detectors creates a maximum of 8 different voltage settings. In one embodiment with arming detector 33 as part of door sensor 30, when the door is open, the state of the arming detector 33 is in a “don't care” condition (X). As such, only 6 states need to be detected. An example encoding of the door switch Door Arming Emergency Voltage A/D (hex Position Switch Shut off Switch Operation (V) code) is a Closed Armed Open Normal 1 AAA Closed Unarmed Open Waiting 0.82 8BF for Arm Closed Armed Closed Shutdown 1.39 ED3 Closed Unarmed Closed Shutdown 1.21 CE8 Open X Open Shutdown 0 000 Open X Closed Shutdown 0.39 428.

Because door sensor 30 communicates such critical information, its time period of transmission may advantageously be more often than UV sensors 21-24 so that the system can react more quickly to an emergency shutoff event. As such, time periods on the order of 1, typically 0.5 to 1.5, seconds are recommended, but not required. Similar to UV sensors 21-24, the transmission time could be selected to be a prime number around 1 second to avoid periodic radio frequency (RF) transmissions with the UV sensor group as previously discussed.

Central computer 11 may also be timing the receipt of the messages from door sensor 30. Should central computer 11 not receive a message from door sensor 30 within some maximum time duration, it will shut down the system so that the operator can determine the cause of the loss of door sensor 30 messages. A system malfunction or a discharged battery may be probable causes.

Another system related issue has to do with the identification of UV sensors as opposed to door sensors. Each sensor may have a unique identification number assigned to it. A rule may be used to easily identify the distinction between a UV sensor and a door sensor. A preferred embodiment uses even identification numbers for the door sensors and odd identification numbers for the UV sensors. However, other coding schemes are not precluded. For example, the UV sensors and door sensors may be associated with any emitter network under central computer command.

An integral part of the sterilization system is the data logging and reporting performed by central computer 11 as shown in FIG. 1. Data logging provides various capabilities to this system, such as recording door open/close events, recording pause/resume events, recording configuration changes, and providing auditing based upon what user is using the system, what he does, and how effectively rooms are disinfected. Further, electricity usage may be monitored in order to detect lamp or ballast failures.

Upon commencing a disinfection job, a data record is created indicating the location being treated and start time. This record is kept in central computer 11, and a copy is transmitted and posted on a secure web server with access only by authorized users with valid access information including a password. Once the first set of UV sensor readings are received by the computer, the estimated time to completion is calculated by the computer based upon the target dosage and the actual incident intensity measurements. An update is then made to the job data record which includes the latest sensor readings, estimated time to completion, and elapsed time. Other data items including starts and stops, job termination, patient identification numbers, and pathogens being treated for are tracked and reported as well. A unique benefit of this reporting system is the ability for sharing of real-time data among different departments of the facility using the system. In the case of a hospital, the admitting department will benefit from such reporting by having the ability to plan when to send patients to a room based upon the estimated job completion time. Or the cleaning department manager can use the real-time data to locate an employee and track their productivity. The infection prevention department may configure reports to aid in determining the efficacy of their disinfection protocol and to track problem patients or areas of their facility. There are other benefits to real-time access to job data and they are not precluded. An additional feature is the ability to post jobs to be performed to this system via a remote web server. That feature affords interested departments the ability to plan and schedule resources and to plan patient room assignments.

The system employs an Internet Protocol address sharing scheme which enables two or more wireless devices shown in FIG. 1, central computer 11 and remote control 40, the ability to communicate with each other inside or behind a firewall. Typically, the devices acquire an IP address dynamically from the hosting DHCP server so none of the devices can predict the address of any other device. The devices are capable of communicating with the Internet through the firewall, but their local network addresses are translated as their messages are sent out to the Internet. The firewall does not accept incoming traffic except in response to an outbound message.

There is no local name-server in which the devices can store their names and network addresses. The problem becomes a question of how the devices find each other so they can communicate. Within the present disclosure, this may be solved by having central computer 11 capture and send its local untranslated IP address to a remote Internet host. As the message is sent, the address of central computer 11 is translated (encapsulated) by the firewall, but the message payload contains the local untranslated IP address given by the DHCP server inside the firewall. Remote control 40 contacts the remote Internet host and reads the local untranslated IP address of the computer 11. Remote control 40 can now directly contact central computer 11 using the local untranslated IP address on the local network inside the firewall. Although the Internet host is outside the firewall and cannot use the untranslated address directly, it can store the address that is scoped to the inside-the-firewall devices. Other inside-the-firewall devices can use the Internet host as a name-server for local addresses.

In addition to allowing these devices the ability to communicate with each other, the host facility's firewall automatically blocks all attempts to communicate with them from devices located outside the firewall. All addresses are automatically updated to the remote Internet host any time a new IP address is obtained.

The central computer of the present disclosure may include its own web server, which may be accessed remotely. This allows an authenticated remote user to access local data and even remotely control the system.

In some embodiments, the devices may communicate with the Internet via a wireless telephone network (e.g., a cellular network). This may be advantageous in situations where it is not desirable to rely on the Internet connectivity of the facility.

The Internet connection, in whatever form, is also useful for such purposes as job reporting, remote system configuration, data backup, diagnostics, and remote operation.

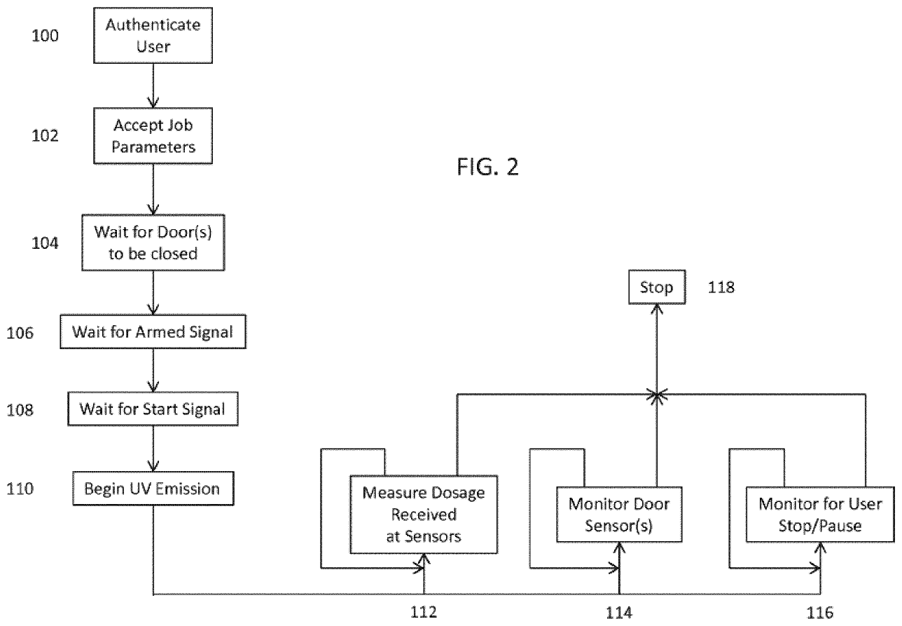

FIG. 2 presents a flowchart of one embodiment of how a device according to the present disclosure may be operated. At step 100, the user is authenticated via username and password, or any other suitable authentication means, at the remote control (or via an on-site or off-site computer).

At step 102, the user selects various job parameters to configure the system. These parameters may include such items as a name or numerical designator for the room/area being disinfected, a desired UV dosage based on the disinfection level desired and the pathogen(s) to be killed, and what set of peripherals to use in the cleaning. These peripherals include things like the door sensor(s) and UV sensor(s).

At steps 104, 106, and 108 the system waits for the door closed signal(s), armed signal, and start signal to occur, in order. Then at step 110, UV irradiation commences.

While the UV irradiation is in process, the system undergoes three loops: measuring the dosage received at the sensors 112, monitoring the door sensor 114 (which includes the emergency stop button on the door sensor), and monitoring for a user stop/pause instruction 116. When any of these monitoring loops detects its condition, UV irradiation is ceased. The three monitoring loops may advantageously occur at different frequencies. For example, the door monitoring loop is a safety concern, so it may be advantageous for it to occur at a higher frequency than the dosage monitoring loop. The user stop/pause monitoring loop may or may not be implemented as a listening loop; alternatively, it may simply be a command from the user that directly interrupts the UV irradiation. All of these embodiments are within the scope of the present disclosure.

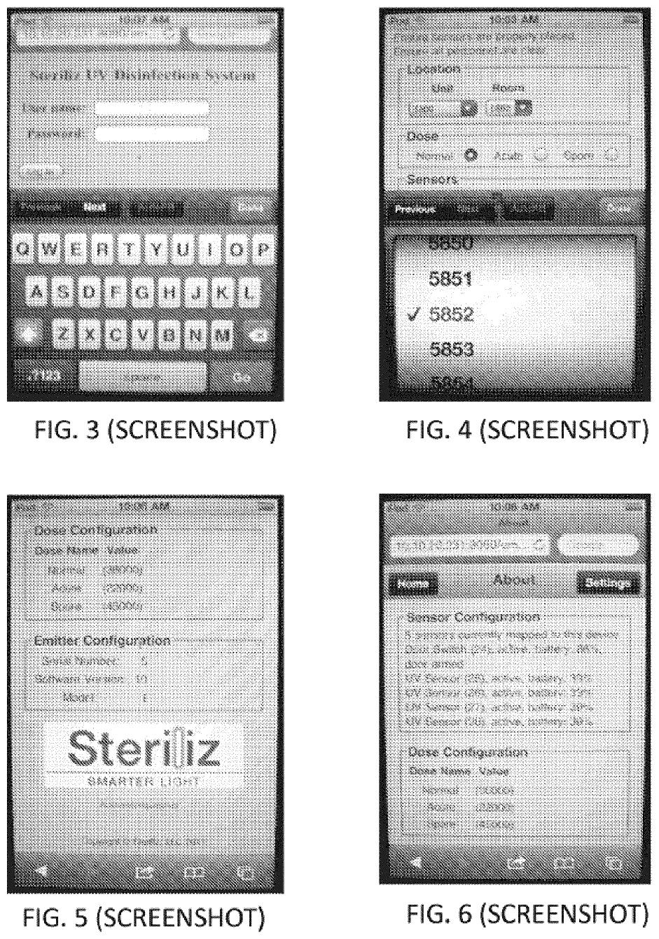

FIGS. 3-11 show screenshots of some of the screens that may be displayed on the remote control of the present disclosure. In the embodiment shown, this software is running on Apple iOS. However, the remote control may provide the same or similar functionality through any of a variety of software platforms.

FIG. 3 shows a Login screen, allowing a user to authenticate via username and password. FIG. 4 shows a Disinfect screen that allows the user to select the Unit, the room being cleaned, and the dosage desired. Rooms may be pre-programmed into the system for easy logging and auditing of cleaning procedures and then selected via drop down menus. One embodiment includes automatic position detection, via GPS, inertial positioning, Wi-Fi triangulation, or other suitable methods. The logging of treatment records allows 2D/3D visualizations of things such as untreated areas, areas with high treatment frequency, etc.

FIG. 5 shows the dose and emitter configuration. FIG. 6 shows an About screen, which lists the active sensors and their battery states.

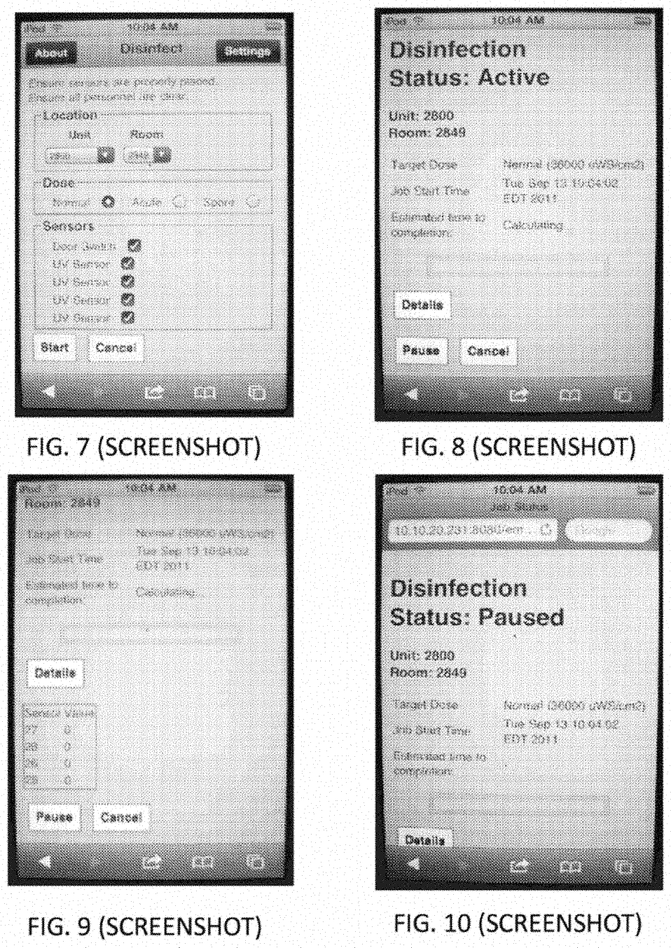

FIG. 7 shows the Disinfect screen, ready to start. At least one UV sensor must be enabled in order for the system to operate.

FIG. 8 shows a Status page listing the parameters of the disinfection job and the estimated time to completion, which the system may calculate based on the UV light levels observed at the UV sensors.

FIG. 9 shows the detail view available in FIG. 8, demonstrating the levels observed at each sensor.

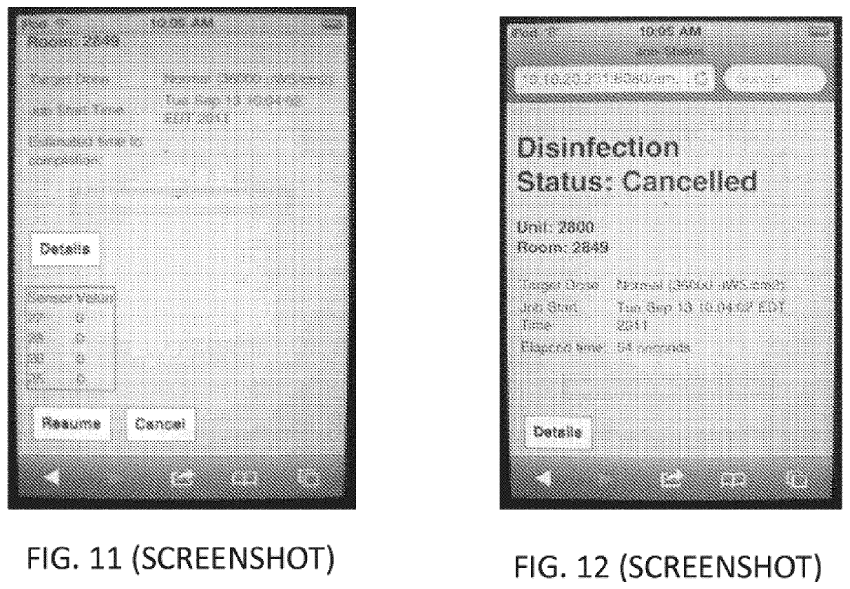

FIG. 10 shows the Status screen of a paused disinfection job, and FIG. 11 shows the option to resume or cancel the paused job. The job may be paused or canceled at any time by the operator via the remote control, so long as there is network connectivity between the remote control and the emitter. If connectivity is lost, the job may be stopped by pressing the emergency stop switch on the door safety sensor. FIG. 12 shows the “Status page” for a job that has been canceled.

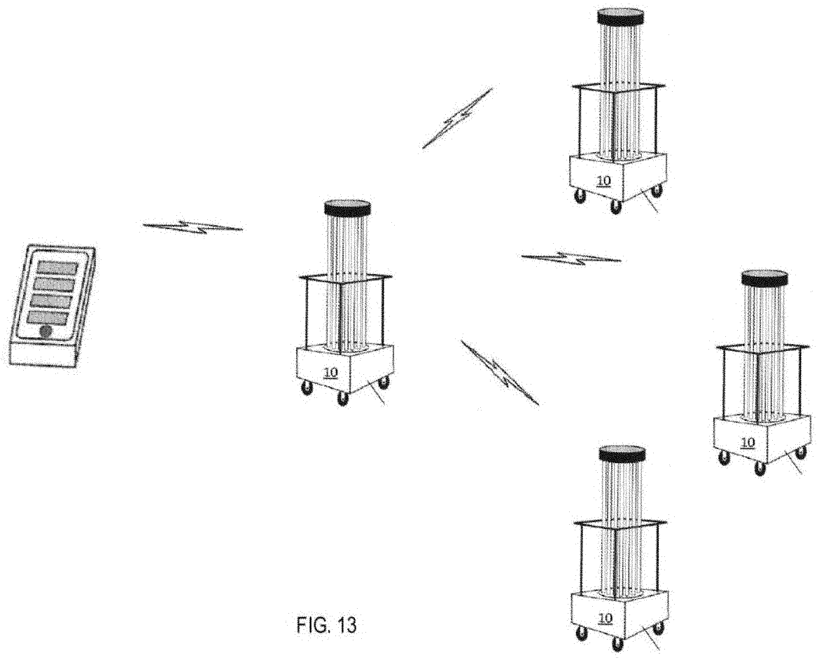

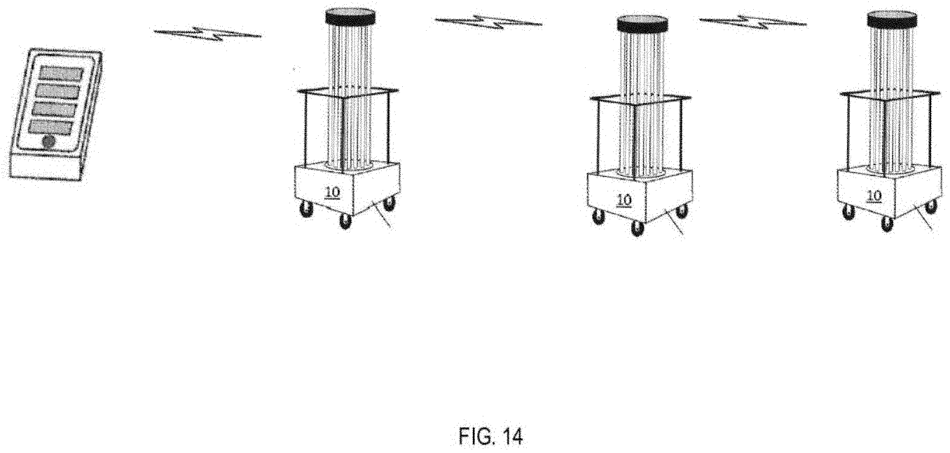

A preferred embodiment of the present invention broadly embodied in FIGS. 13 and 14 includes a multiple emitter configuration, with a Master emitter 10a, and plural Slave emitters 10b.

The remotely located control station for operation of this system is in data communication with only the Master emitter 10a. The multiple slave emitters 10b are not in data communication with the control station. The slave emitters 10b are separately in data communication only with the Master emitter 10a, not with the control station. With this embodiment many Slave emitters 10b may be located within a large space to be disinfected and by employing a repeater technique, the distance limitation is eliminated. The repeater technique employed may be of several different types. The preferred embodiment utilizes a simple repeat of a received radio frequency signal one time by each emitter that receives the signal. In this design a quasi-mesh network is established. There is no back-and-forth signal transmission between the Slave emitters 10b. When a Slave emitter 10b receives a signal meant for another Slave emitter 10b, not for itself it simply re-broadcasts it. In this way a Slave emitter 10b located out of range of the Master emitter 10a will receive a message intended for it through the process of the Slave emitters 10b sequentially re-broadcasting the message one time until the Slave emitter 10b for which the message is intended receives it.

The present invention is capable of detecting and correcting a potential overload condition. The preferred embodiment utilizes a voltage drop monitoring scheme. When the Master emitter 10a is fully energized it measures the AC voltage to which it is connected, and the AC current it is drawing. The Master emitter 10a then sends a message to one of the Slave emitters 10b instructing it to begin energizing its lamps. If the Master emitter 10a measures a significant subsequent drop in voltage or increase in current, it indicates a potential that two emitters are plugged into the same circuit, posing an overload condition. In such an event the Master emitter 10a will transmit a signal to the Slave emitter 10b instructing it to turn its lamps off and not participate in the current disinfection job. This process is repeated for each Slave emitter 10b as it begins to energize its respective lamps. Each already energized emitter will participate in the monitoring scheme and advise as needed.

The present invention incorporates many other features to ensure safe and reliable operation. These features include, but are not limited to the following:

A password-protected remote control, which will only operate the system after a valid user name and password has been entered, prevents unauthorized use of the device.

A fail-safe lamp operating time limit also prevents damage to the lamps and overexposure of the room being treated. The present invention includes a built-in timer limiting the “on time” for a treatment cycle. This may come initially programmed to limit the maximum lamp “on time” to approximately one hour. It may be changed to any amount of time up to, for example, two hours by an authorized service technician. In the unlikely event of a computer failure, the built-in timer will turn off the lamps after the lamps have been on for the programmed amount of time, even if the job hasn't completed.

The present invention also may incorporate an internal circuit breaker that will turn off all power to the system in the unlikely event of an overload that is not protected by the facility's circuit protection system. In some embodiments, this may be a 20-amp time delay circuit breaker. This circuit breaker must be manually reset by a qualified technician. To protect against fires and thermal damage, the system may include an internal temperature sensor that will turn off all power in the event the internal temperature of the lower electronics cabinet is at or above the designed temperature limit.

The present invention also may be equipped with a battery conservation system that automatically turns off power to on-board battery-operated devices (including chargers for remote sensors, cooling fan, and computer) when it has not been plugged into facility electrical power for a period of time. It is recommended that the device be plugged into facility electrical power when not in use.

The UV emitters 10 may be equipped with two wheels that have push down brakes which, when depressed and locked into place, prevent them from rotating and spinning. The other two wheels are free to rotate and spin. They may be locked such that they can only spin and not rotate, like the rear wheels on a shopping cart.

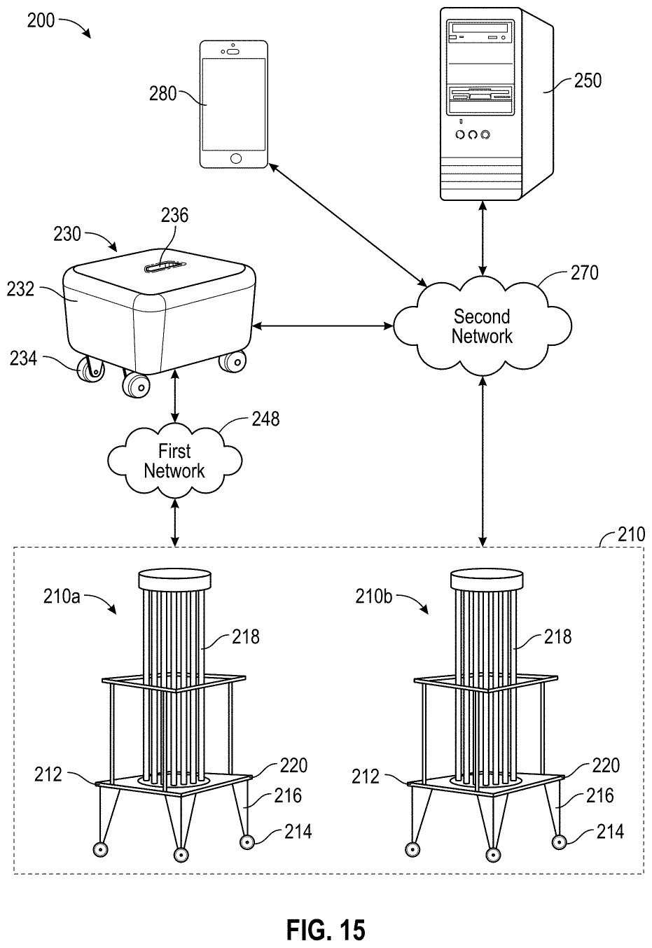

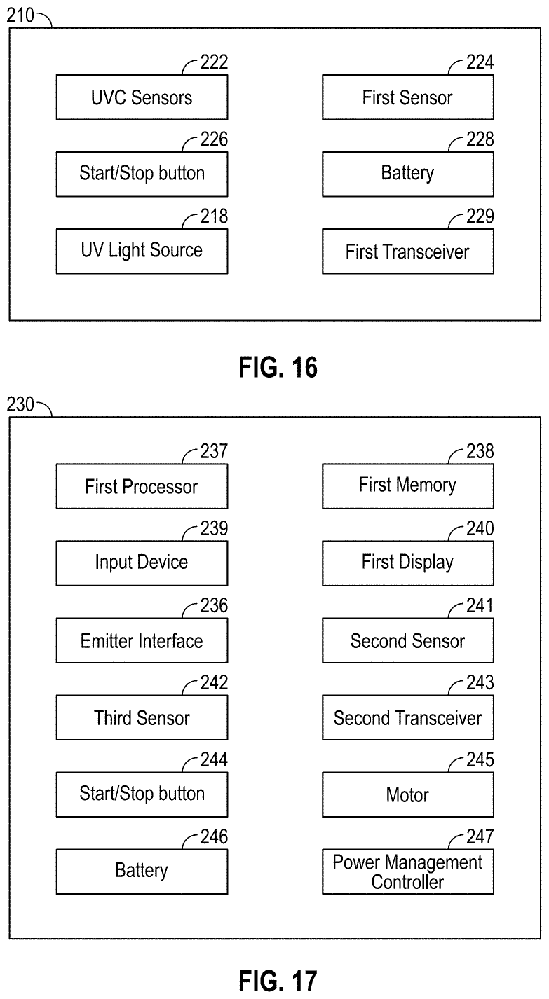

In one embodiment, the present invention discloses a sterilization system for improving duty cycle of the robotic system and the Ultraviolet (UV) emitter modules disinfecting closed environment. FIG. 15 shows an environment in which sterilization system 200 is used, in accordance with one embodiment of the present invention. Sterilization system 200 includes a plurality of UV emitter modules 210, e.g., first UV emitter module 210a, second UV emitter module 210b and so on, collectively referred to as UV emitter modules 210. Each of UV emitter modules 210 includes base 212. Base 212 may be made of suitable material and may be used for placing Ultraviolet (UV) light source 218. Base 212 may be provided with wheels 214 via caskets or support frames 216. Further, UV emitter module 210 may include UV light source 218. UV light source 218 may indicate a UV germicidal bulb that emits very short ultraviolet wavelengths from 100 to 280 nanometers that damages the Deoxyribonucleic Acid (DNA) of bacteria, viruses, and other pathogens. Further, UV emitter module 210 includes housing 220 provided at base 212. Housing 220 may include one or more Ultraviolet C (UVC) sensors 222 (FIG. 16) configured for measuring power or intensity of incident UV radiation. Further, housing 220 may include first sensor 224. First sensor 224 may include, but not limited to, an image sensor, infrared sensor, laser sensor, motion sensor configured to detect the presence of humans in the vicinity of UV emitter module 210. In one embodiment, UV emitter module 210 may comprise a start/stop button 226 provided at the outer side of housing 220 allowing the user to operate UV emitter 210. Further, UV emitter module 210 may include a battery 228 for powering UV light source 218. In the current embodiment, UV emitter module 210 may include a first transceiver 229 for transmitting or receiving signals from external devices such as a motorized base or robotic system 230, or server 250, or user device 280.

Sterilization system 200 includes robotic system 230 communicatively connected to UV emitter module 210 via first network 248. Robotic system 230 includes housing 232 made of a variety of materials such as metal, plastic or any other suitable material. Housing 232 may be provided in a variety of shapes such as square, rectangular or any other shape. Housing 232 includes wheels 234 provided at the bottom for maneuvering robotic system 230 over surface or ground. Further, robotic system 230 includes emitter interface 236 configured for connecting robotic system 230 to UV emitter for transporting and charging battery 246 via UV emitter module 210.

FIG. 17 shows a block diagram of robotic system 230. Robotic system 230 includes first processor 237, first memory 238, input device 239, first display 240, second sensor 241, third sensor 242, second transceiver 243, start/stop button 244, motor 245, battery 246, and power management controller 247.

First processor 237 includes a central processing unit (CPU), graphics-processing unit (GPU) or both. First processor 237 may include any suitable processing device, such as microprocessor, microcontroller, integrated circuit, logic device, or other suitable processing device.

First memory 238 communicates with first processor 237 via bus (not shown). First memory 238 may include one or more computer-readable media, including, but not limited to, non-transitory computer-readable media, RAM, ROM, hard drives, flash drives, or other memory devices. First memory 238 may store information accessible by first processor 237, including computer-readable instructions that may be executed by first processor 237. In one example, first memory 238 may be used to store data that may be retrieved, manipulated, created, or stored by first processor 237.

Instructions can be any set of instructions that when executed by first processor 237, cause first processor 237 to perform certain operations. Instructions may also reside, completely or at least partially, within first memory 238 and/or within first processor 237 during execution thereof by robotic 230, first memory 238 and first processor 237 also constituting machine-readable medium. Instructions may further be transmitted or received over first network 238 or second network 270 via second transceiver 243 utilizing one of several well-known transfer protocols or custom protocols.

Input device 239 includes keyboard/keypad, or touch screen used for providing an input to robotic system 230.

First display 240 includes a device used for displaying data in the form of text/video to users.

Second sensor 241 may include an image sensor configured to detect or capture presence of users or objects in the vicinity of robotic system 230 or within the closed environment. In one example, second sensor 241 may include optical, infrared, or laser detection sensor configured to detect position or motion or movement of users in the vicinity of robotic system 230.

Third sensor 242 may include location or position sensor configured for determining location of robotic system 230. In one example, third sensor 242 may include a Global Positioning System (GPS) sensor.

Second transceiver 243 sends and receives data from robotic system 230 to other devices such as UV emitter module 210, server 250, or user device 280 via first network 248 or second network 270.

Start/stop button 244 includes a physical button provided at the outer surface of housing 232 or soft touch button provided on first display 240. Start/stop button 244 may be configured to power ON or power OFF robotic system 230.

Motor 245 may include a DC motor configured to drive wheels 234 when instructed by first processor 237.

Battery 246 acts as a power source for operating motor 245 or various components of robotic system 230. Battery 246 may include a standalone rechargeable battery or battery having an electric cord plugged to an energy source (AC/DC).

Power management controller 247 includes a scheme or set of instructions configured to enable robotic system 230 to turn OFF itself when not used for a predetermined time period for reducing dynamic power consumption of battery 246. Further, scheme is configured to re-enable robotic system 230 with a fast wake-up time of less than e.g., 1 millisecond (ms), when the user presses start/stop button 244.

Although it is shown that housing 232 includes second sensor 241 and third sensor 242, person skilled in the art will appreciate that second sensor 241 and third sensor 242 may be provided at the outer side of housing 232 to capture presence of users or to detect location of robotic system 230 and transmit signals to first processor 237 for processing the data sensed by second sensor 241 and third sensor 242.

As specified above, UV emitter module 210 is communicatively connected to robotic system 230 via first network 248. First network 248 may include a short-range wireless network such as Bluetooth, ZigBee, Radio-frequency Identification (RFID), beacons, Near Field Communication (NFC) and so on. Alternatively, first network 248 may include wireless network, wired network or combination thereof. First network 248 may be implemented as one of the different types of networks, such as intranet, local area network (LAN), wide area network (WAN), the internet, and the like. First network 248 may either be a dedicated network or shared network. Shared network represents an association of the different types of networks that use variety of protocols, for example, Hypertext Transfer Protocol (HTTP), Transmission Control Protocol/Internet Protocol (TCP/IP), Wireless Application Protocol (WAP), and the like, to communicate with one another.

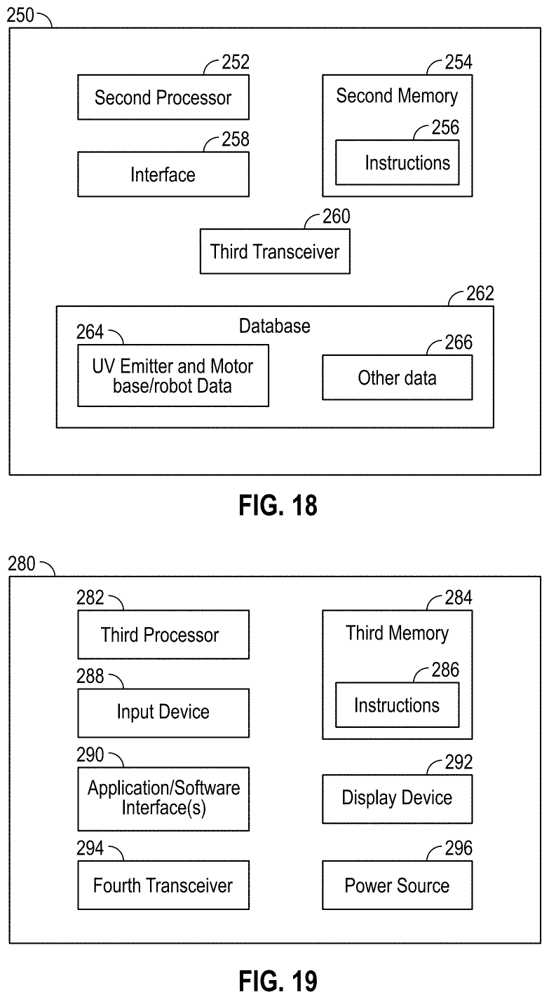

Further, robotic system 230 is communicatively connected to server 250 via second network 270. FIG. 18 shows a block diagram of server 250. Server 250 includes second processor 252 (e.g., central processing unit (CPU), graphics processing unit (GPU) or both. Server 250 includes second memory 254 which communicates with second processor 252 via bus (not shown).

Second processor 252 may include any suitable processing device, such as a microprocessor, microcontroller, integrated circuit, logic device, or other suitable processing device.

Second memory 254 may include one or more computer-readable media, including, but not limited to, non-transitory computer-readable media, RAM, ROM, hard drives, flash drives, or other memory devices. Second memory 254 may store information accessible by second processor 252, including computer-readable instructions 256 that can be executed by second processor 252. In one example, second memory 254 can be used to store data that can be retrieved, manipulated, created, or stored by second processor 252.

Instructions 256 can be any set of instructions that when executed by second processor 252, cause second processor 252 to perform operations. Instructions 256 may also reside, completely or at least partially, within second memory 254 and/or within second processor 252 during execution thereof by server 250, second memory 254 and second processor 252 also constituting machine-readable medium. Instructions 256 may further be transmitted or received over second network 270 via third transceiver 260 utilizing one of several well-known transfer protocols or a custom protocol.

Server 250 includes user interface (UI) 258 i.e., software or Application interface allowing the user of server 250 to interact with user device 280, UV emitter module 210 and robotic system 230.

Server 250 further includes a third transceiver 260 configured to send and receive data from server 250 to other devices such as robotic system 230, user device 280 and UV emitter module 210.

Server 250 further includes database 252. Database 252 indicates data structure configured for storing the information. In the current embodiment, database 252 includes UV emitter and motor base or robotic system data 264, and other data 166. UV emitter and motor base or robotic system data 264 includes data corresponding to UV emitter module 210 and robotic system 230. For example, UV emitter and motor base or robotic system data 264 includes name, type, serial number or unique identification number, manufacturer, manufacturing date, model number, maintenance schedule, location, etc. of each of UV emitter modules 210 i.e., first UV emitter module 210a and second UV emitter module 210b and robotic system 230.

Second network 270 may include wireless network, wired network or combination thereof. Second network 270 can be implemented as one of the different types of networks, such as intranet, local area network (LAN), wide area network (WAN), the internet, and the like. Second network 270 may either be a dedicated network or shared network. Shared network represents an association of different types of networks that use variety of protocols, for example, Hypertext Transfer Protocol (HTTP), Transmission Control Protocol/Internet Protocol (TCP/IP), Wireless Application Protocol (WAP), and the like, to communicate with one another. Further, second network 270 may include a variety of network devices, including routers, bridges, servers, computing devices, storage devices, and the like.

Further, robotic system 230 and UV emitter module 210 are further communicatively connected to user device 280 via second network 270. FIG. 19 shows a block diagram of user device 280. User device 280 may include mobile phone, laptop, desktop, tablet, smart watch and any other electronic device. User device 280 includes third processor 282 (e.g., central processing unit (CPU), graphics processing unit (GPU) or both. User device 280 includes third memory 284 which communicates with third processor 282 via bus (not shown).

Third processor 282 may include any suitable processing device, such as a microprocessor, microcontroller, integrated circuit, logic device, or other suitable processing device.

Third memory 284 may include one or more computer-readable media, including, but not limited to, non-transitory computer-readable media, RAM, ROM, hard drives, flash drives, or other memory devices. Third memory 284 may store information accessible by third processor 282, including computer-readable instructions 286 that can be executed by second processor 252. In one example, third memory 284 can be used to store data that can be retrieved, manipulated, created, or stored by third processor 282.

Instructions 286 can be any set of instructions that when executed by third processor 282, cause third processor 282 to perform operations. Instructions 286 may also reside, completely or at least partially, within third memory 284 and/or within third processor 282 during execution thereof by user device 280, third memory 284 and third processor 282 also constituting machine-readable medium. Instructions 286 may further be transmitted or received over second network 270 via fourth transceiver 294 utilizing one of several well-known transfer protocols or a custom protocol.

User device 280 includes input device 288. Input device 288 includes keyboard/keypad, or touch screen used for providing an input to user device 280.

User device 280 includes Application or software interface allowing the user of user device 280 to interact with UV emitter module 210, robotic system 230, and server 250.

User device 280 further includes display device 292 used for displaying data in the form of text/video to user of user device 280.

User device 280 further includes fourth transceiver 294 configured to send and receive data from user device 280 to other devices such as UV emitter module 210, robotic system 230, and server 250.

User device 280 further includes power source 296 such as a battery for powering various components of user device 280.

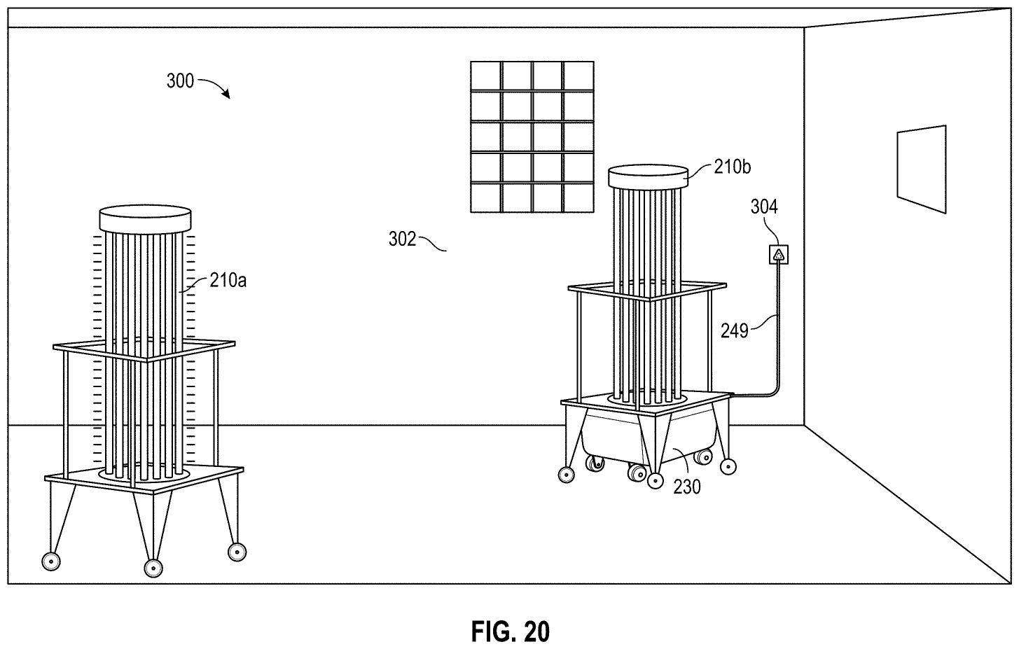

Now referring to FIGS. 15, and 20 through 23, the operational features and interaction of UV emitter module 210, robotic system 230, server 250 and user device 280 are explained. In accordance with one embodiment of the present invention, UV emitter modules 210 i.e., first UV emitter module 210a and second UV emitter module 210b may be used for sterilizing or disinfecting surfaces within closed environment 300 as shown in FIG. 20. In one embodiment, each of first UV emitter module 210a and second UV emitter module 210b may be provided with a unique identification number. Further, robotic system 230 may be configured to store the unique identification number associated with first UV emitter module 210a and second UV emitter module 210b in first memory 238. Referring to FIG. 20, closed environment 300 such as a room, hospital or container where sterilization system 200 including separate UV emitter modules i.e., first UV emitter module 210a and second UV emitter module 210b, and robotic system 230 can be implemented is shown, in accordance with one exemplary embodiment of the present invention. Closed environment 300 may include wall 302 for mounting charging dock or power source 304. In the current embodiment, first UV emitter module 210a and second UV emitter module 210b, and robotic system 230 may be used as separate devices, each having their own resources such as battery for performing their respective operations. For example, consider battery 228 of first UV emitter module 210a is fully charged and being used for disinfecting the interior or surface of the closed environment. Further, consider battery 228 of second UV emitter module 210b is fully discharged. In accordance with current embodiment, robotic system 230 may mount second UV emitter module 210b via emitter interface 236 and maneuver second UV emitter module 210b to charging dock 304 as shown in FIG. 20.

In order to charge battery 228, each of first UV emitter module 210a and second UV emitter module 210b may be provided with cord 249 extending from housing 220. Alternatively, robotic system 230 may be provided with cord 249. When robotic system 230 mounted with second UV emitter module 210b reaches charging dock 304, cord 249 may be connected to charging dock 304 for charging second UV emitter module 210b as shown in FIG. 20.

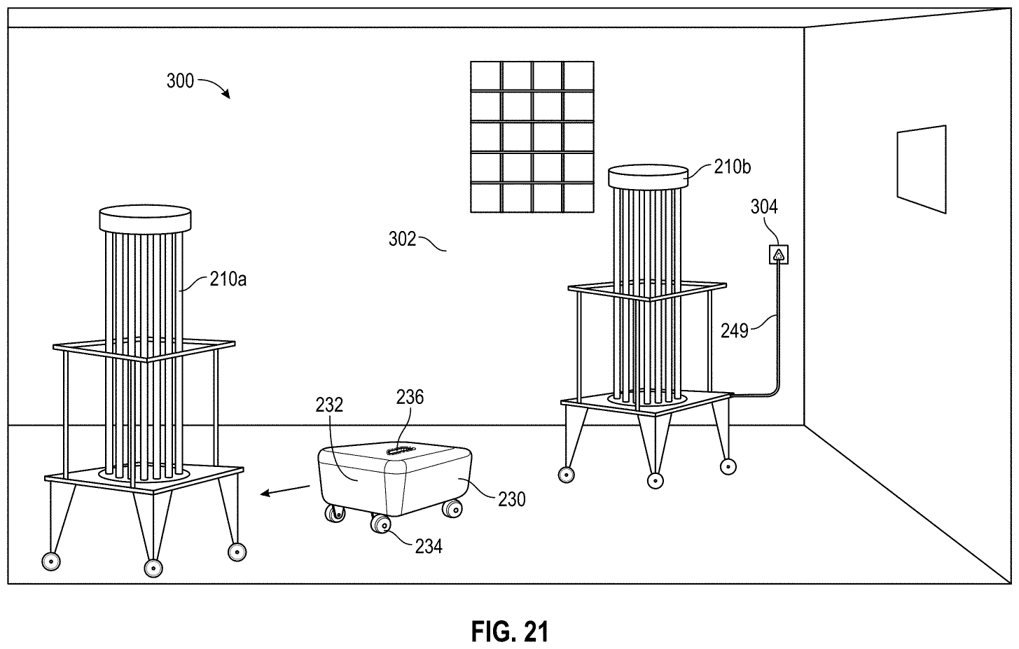

Now, consider that battery 228 of first UV emitter module 210a is fully discharged. In such a scenario, first UV emitter module 210a may transmit a signal indicating discharge of its battery 228. In the current embodiment, robotic system 230 may be configured to store location of each of first UV emitter module 210a, second UV emitter module 210b and charging dock 304 in first memory 238. As such, when first UV emitter module 210a transmits signal indicating discharge of battery 228, second transceiver 243 may receive the signal and send the signal to first processor 237. Subsequently, first processor 237 may employ emitter interface 236 to detach from second UV emitter module 210b and instruct motor 245 to maneuver wheels 234 to reach location of first UV emitter module 210a as shown in FIG. 21.

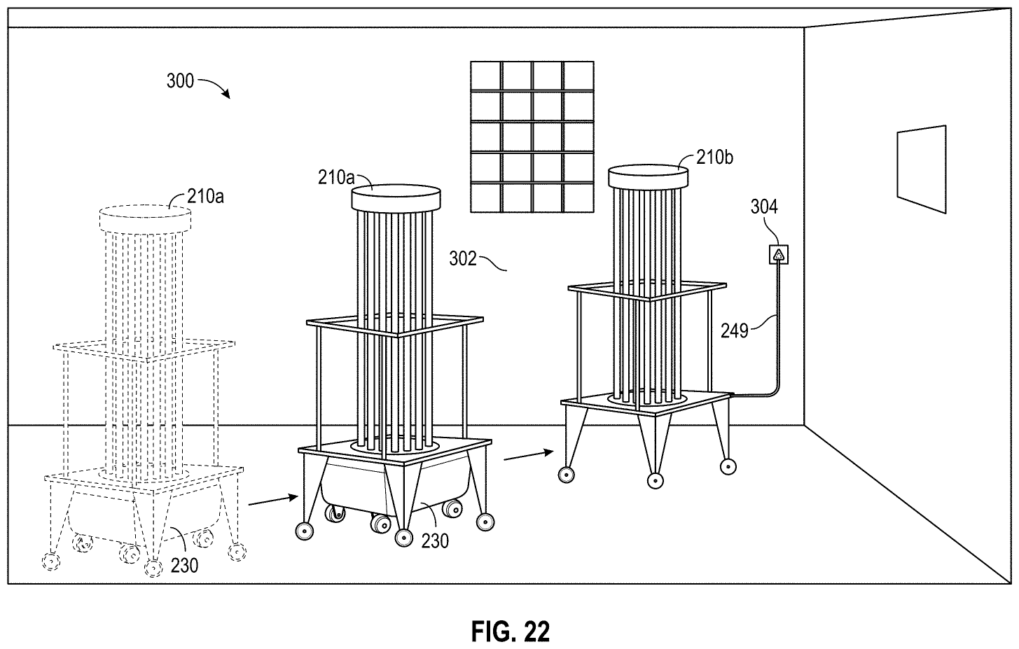

After reaching the location of first UV emitter module 210a, robotic system 230 may employ emitter interface 236 for mounting first UV emitter module 210a to robotic system 230. Subsequently, robotic system 230 may maneuver first UV emitter module 210a to reach charging dock 304 as shown in FIG. 22.

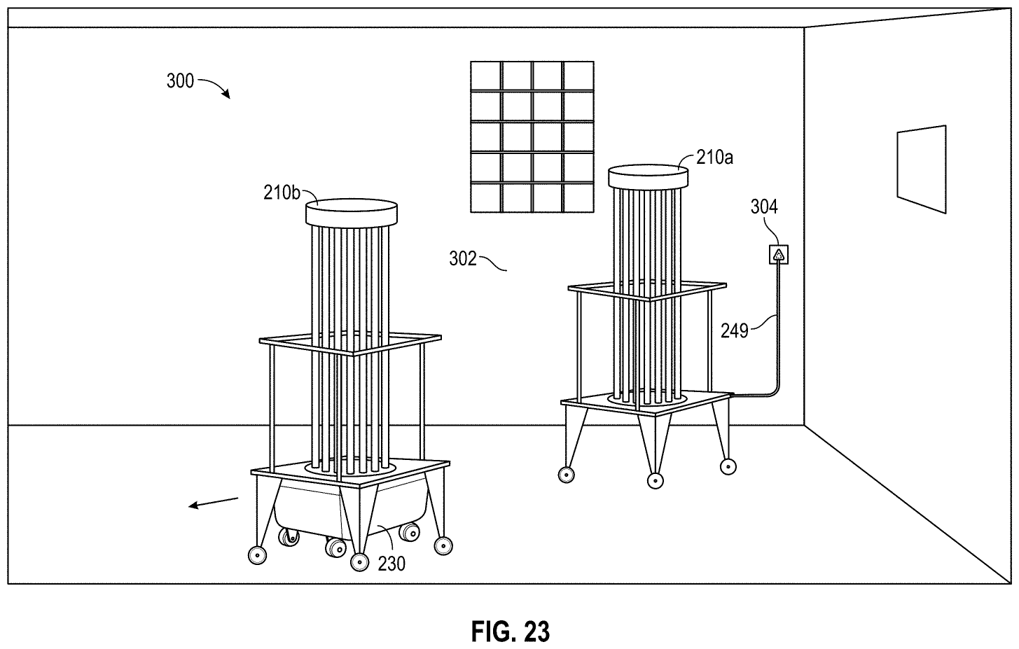

After reaching charging dock 304, robotic system 230 may demount first UV emitter module 210a and mount second UV emitter module 210b to move aside second UV emitter module 210b from charging dock 304. Subsequently, robotic system 230 may mount first UV emitter module 210a and maneuver closer to charging dock 304 for connecting cord 249 to charging dock 304 in order to charge battery 228 of first UV emitter module 210a, as shown in FIG. 23. After connecting first UV emitter module 210a to charging dock 304, robotic system 230 may mount second UV emitter module 210b and maneuver second UV emitter module 210b to a desired area or location (FIG. 23) within closed environment 300 where disinfection needs to be performed.

In accordance with one embodiment of the present invention, when second UV emitter module 210b is connected to charging dock 304 for charging battery 228 (FIG. 20), battery 246 of robotic system 230 may be charged through emitter interface 236. This way, both second UV emitter module 210b and robotic system 230 may be charged while first UV emitter module 210a is used for disinfecting closed environment 300. Similarly, when first UV emitter module 210a is connected to charging dock 304 for charging battery 228, battery 246 of robotic system 230 may be charged through emitter interface 236. This way, both first UV emitter module 210a and robotic system 230 may be charged while second UV emitter module 210b is used for disinfecting closed environment 300.

In another embodiment, battery 246 of robotic system 230 may be charged or recharged when robotic system 230 is transporting or maneuvering second UV emitter module 210b after charging battery 228 of second UV emitter module 210b, as shown in FIG. 23. Similarly, battery 246 of robotic system 230 may be charged or recharged when robotic system 230 is transporting or maneuvering first UV emitter module 210a after charging battery 228 of first UV emitter module 210a. This way, battery 246 of robotic system 230 may get charged continuously and be ready to transport UV emitter module 210 to charging dock 304 immediately after receiving signal indicating discharge of battery 228. In other words, battery 246 of robotic system 230 may get continuously charged or recharged from connected UV emitter modules 210, thereby allowing robotic system 230 and UV emitter modules 210 to have a 100% duty cycle. This ensures uninterrupted operation of robotic system 230 and UV emitter modules 210 for disinfecting closed environment 300 using UV emitter modules 210 and saves cost and man hours for users.

As specified above, robotic system 230 includes power management controller 247. In one implementation, power management controller 247 may be configured to turn OFF robotic system 230 after a hiatus, say 2 minutes of non-use or disconnection from UV emitter modules 210 to prolong life of battery 246. When one of first UV emitter module 210a and second UV emitter module 210b transmits a signal corresponding to discharge of battery 228, then second transceiver 243 may receive the signal. Concurrently, power management controller 247 may turn ON robotic system 230. Subsequently, first processor 237 may employ third sensor 242 to detect location of first UV emitter module 210a or second UV emitter module 210b that transmitted signal corresponding to discharge of battery 228. It should be understood that robotic system 230 may employ first processor 237 to identify whether first UV emitter module 210a or second UV emitter module 210b has transmitted the signal using their unique identification number inferred from the signal. Consider that first processor 237 detects that second UV emitter module 210b transmitted signal indicating discharge of battery 228. Subsequently, robotic system 230 may employ third sensor 242 to detect the location of second UV emitter module 210b. After detecting the location, first processor 237 may employ second sensor 241 to detect presence of humans or objects in vicinity or path to reach the location of second UV emitter module 210b. Further, first processor 237 may employ motor 245 to maneuver wheels 234 to reach the location of second UV emitter module 210b. After reaching the location of second UV emitter module 210b, first processor 237 may employ emitter interface 236 to mount second UV emitter module 210b. Subsequently, first processor 237 may employ first sensor 241 and second sensor 242 to maneuver robotic system 230 mounted with second UV emitter module 210b to charging dock 304, as explained above with the help of FIGS. 20 to 23.

In one exemplary embodiment, robotic system 230 may be pre-configured to maneuver to one of first UV emitter module 210a and second UV emitter module 210b and transport to charging dock 304 for charging battery 228 at predetermined time intervals, and subsequently to predefined area to disinfect or sterilize using UV light source 218. Considering that battery 228 of first UV emitter module 210a takes about 30 minutes to charge fully and takes one (1) hour to discharge battery 228 when used for operating UV light source 218 for disinfecting, then robotic system 230 may be configured to maneuver to first UV emitter module 210a at every one hour thirty minutes to take it to charging dock 304 for charging battery 228 and subsequently to predefined area for disinfecting the predefined area. Similarly, the robotic system 230 may be pre-configured to maneuver second UV emitter module 210b to charging dock 304 for charging battery 228 at predetermined time intervals and then maneuver to predefined locations.

In another embodiment, user of user device 280 may provide instructions to robotic system 230 to mount and maneuver one of the first UV emitter module 210a and second UV emitter module 210b to charging dock 304. Similarly, user device 280 may be used to instruct robotic system 230 to mount and maneuver one of the first UV emitter module 210a and second UV emitter module 210b from charging dock 304 to a desired area for disinfecting closed environment 300. In accordance with current embodiment, user of user device 280 may be able to selectively instruct robotic system 230 to maneuver one of the first UV emitter module 210a and second UV emitter module 210b for charging them or disinfecting closed environment 300. In one example, first UV emitter module 210a and second UV emitter module 210b may be provided in different sizes. For instance, first UV emitter module 210a may be provided in a smaller configuration than second UV emitter module 210b. In another example, first UV emitter module 210a and second UV emitter module 210b may be provided different UV frequencies such as UV-C or far-UV or even alternative disinfection technology such as an electrostatic fogger. As such, the user may use user device 280 to selectively instruct robotic system 230 to maneuver one of first UV emitter module 210a and second UV emitter module 210b to disinfect different areas in closed environment 300. For example, the user may use user device 280 to selectively instruct robotic system 230 to maneuver first UV emitter module 210a to disinfect areas such as corners of closed environment 300. This way, the user may use robotic system 230 to return a discharged UV emitter module to a charging dock and leave it there to charge and immediately pick up a fully charged UV emitter module and return to desired area for disinfecting surface, thereby improving overall duty cycle of robotic system and UV emitter modules.

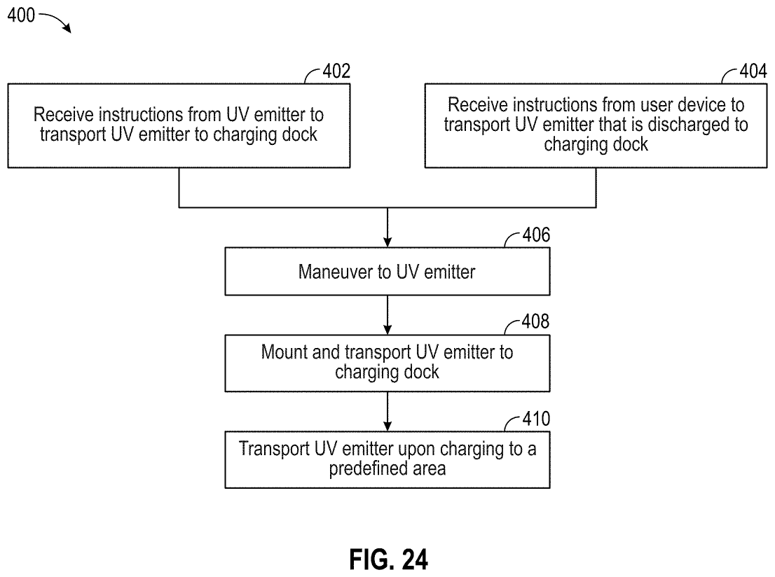

Now referring to FIG. 24, a method 400 of improving the duty cycle of robotic system and UV emitter modules is explained, in accordance with one exemplary embodiment of the present invention. The order in which method 400 is described should not be construed as a limitation, and any number of the described method blocks can be combined in any order to implement method 400 or alternate methods. Additionally, individual blocks may be deleted from method 400 without departing from the spirit and scope of the subject matter described herein. Furthermore, method 400 may be implemented in any suitable hardware, software, firmware, or combination thereof. However, for ease of explanation, in the embodiments described below, method 400 may be implemented using the above-described robotic system 230.

As specified above, robotic system 230 may be configured to receive instructions or signals from one of first UV emitter module 210a and second UV emitter module 210b. Alternatively, robotic system 230 may be configured to receive instructions from the user of user device 280. Alternatively, robotic system 230 may be pre-configured to maneuver to one of first UV emitter module 210a and second UV emitter module 210b and take it to charging dock 304 and then maneuver to predefined area at predefined time intervals. As such, robotic system 230 may receive instructions or signals from UV emitter e.g., first UV emitter module 210a to transport first UV emitter module 210a to charging dock 304, as shown at step 402.

Alternatively, robotic system 230 may receive instructions from user device to UV emitter e.g., first UV emitter module 210a to transport first UV emitter module 210a to charging dock 304, as shown at step 404.

After receiving the instructions (step 402 or step 404), robotic system 230 may employ first processor 237 to employ motor 245. Further, robotic system 230 may employ second sensor 241 and third sensor 242 for detecting the presence of objects or humans and location of first UV emitter module 210a, respectively. Subsequently, robotic system 230 may maneuver to the location of first UV emitter module 210a, as shown at step 406.

At step 408, robotic system 230 may mount first UV emitter module 210a using emitter interface and transport or maneuver first UV emitter module 210a to charging dock 304.

After charging battery 228 of first UV emitter module 210a, robotic system 230 may transport or maneuver first UV emitter module 210a to a predefined area or location for disinfecting using UV light source 218, as shown at step 410. In accordance with one embodiment of the present invention, when battery 228 of first UV emitter module 210a is getting charged, battery 246 may also get charged through emitter interface 236 of first UV emitter module 210a. After charging battery 228, when robotic system 230 picks up first UV emitter module 210a to take it to the predefined area, battery 246 of robotic system 230 may get charged through emitter interface 236 of first UV emitter module 210a. This ensures that battery 246 of robotic system 230 gets continuously charged or recharged from connected first UV emitter module 210a.

Based on the above, it is evident that the sterilization system including separate Ultraviolet (UV) emitter modules and the robotic system can be used to have a 100% duty cycle for the robotic system and the UV emitter modules. As a result, one robotic system and two UV emitter modules may be used for near 24-hour operation without interruption thereby providing substantial savings in cost to facilities.

As the robotic system and the UV emitter modules are installed with the sensors for detecting presence of the users, they can be operated only when they detect the humans are not present in the closed environment thereby ensuring safety of users and also disinfecting the closed environment.

The sterilization system may be used in hospitals, hotels, food preparation facilities, industrial complexes, shopping malls and any other closed environment.

Although the above disclosure is generally described in which the sterilization system for improving duty cycle of robotic system and Ultraviolet (UV) emitter modules disinfecting closed environment is implemented as a single robotic system and two UV emitter modules, it should be understood that the disclosed sterilization system may be scaled up such that multiple robotic systems can be used in a single facility and multitude of UV emitter modules can be operated to improve sterilization of closed environment and also improve duty cycle of multiple robotic systems and multitude of UV emitter modules.

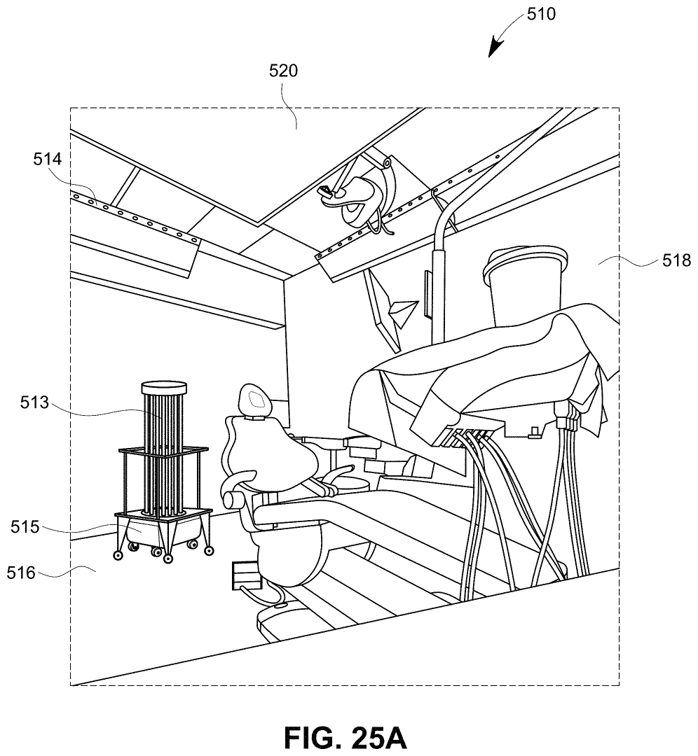

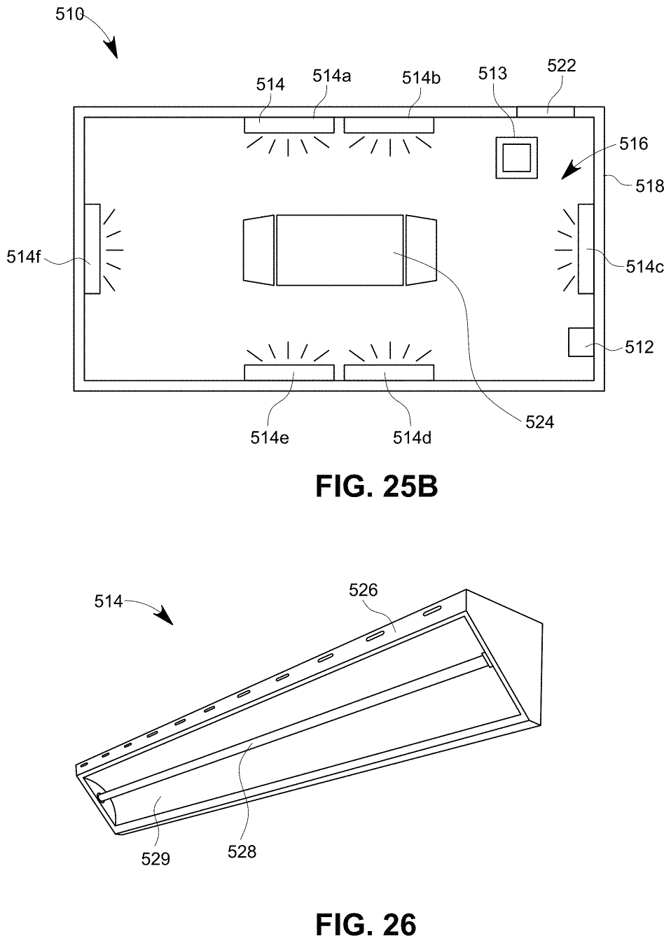

The present invention further discloses a room disinfection system for disinfecting a closed space or surface in the closed space. FIGS. 25A and 25B show an environment 510 in which a room disinfection system or “disinfection system” 512 installs, in accordance with one embodiment of the present invention. Room disinfection system 512 includes at least one UV emitter module 513 placed over a robotic system 515, and plurality of light fixtures or light fixtures 514. UV emitter module 513 includes all the components and operates similar to UV emitter module 210, as explained above. Further, robotic system 515 includes all the components and operates similar to robotic system 230, as explained above. The present embodiment illustrates fixed light fixtures 514 and maneuverable UV emitter module 513 with the help of robotic system 515 to disinfect a closed space 516 or a surface in closed space 516 such as a room.

In one implementation, light fixtures 514 include two or more light fixtures 514. In one example, light fixtures 514 include six light fixtures 514, say a first light fixture 514a, a second light fixture 514b, a third light fixture 514c, a fourth light fixture 514d, a fifth light fixture 514e and a sixth light fixture 514f (collectively referred as light fixtures 514 or simply light fixture 514 when referred to a single light fixture). In one example, light fixtures 514 install in closed space 516 e.g., room/structure having walls 518 and/or ceiling 520, as shown in FIG. 25B.

As can be seen, closed space 516 includes a door 522. In one example, room disinfection system 512 mounts inside closed space 516, as shown in FIG. 25B. In another example, room disinfection system 512 mounts outside closed space 516. In accordance with the present invention, light fixtures 514 mount at walls 518 and/or ceiling 520 such that they align to focus UVC light onto surfaces such as a chair 524 upon which a patient (not shown) rests. Alternatively, light fixtures 514 mount at walls 518 and/or ceiling 520 such that they deliver UVC light to disinfect the entire closed space 516 in a minimum time period (i.e., with maximum efficiency). Here, chair 524 refers to a table or bed in a hospital or clinic. In one example, chair 524 includes a dental chair, medical furniture, medical recliners, and the like.

FIG. 26 shows a feature of light fixture 514, in accordance with one exemplary embodiment of the present invention. Each light fixture 514 includes a frame 526. Frame 526 receives a light source 528. Here, light source 528 connects to frame 526 using a variety of techniques known in the art. In one implementation, frame 526 encompasses a highly polished, precision tuned reflector 529. Reflector 529 helps to maximize UVC output produced by light source 528 and minimizes the time required to disinfect closed space 516 or surface/chair 524.

As specified above, room disinfection system 512 includes six light fixtures 514. Further, each light fixture 514 includes a light source 528. As such, a person skilled in the art understands that each of first light fixture 514a, second light fixture 514b, third light fixture 514c, fourth light fixture 514d, fifth light fixture 514e and sixth light fixture 514f receives a first light source 528a, a second light source 528b, a third light source 528c, a fourth light source 528d, a fifth light source 528e and a sixth light source 528f, respectively. First light source 528a, second light source 528b, third light source 528c, fourth light source 528d, fifth light source 528e and sixth light source 528f are collectively termed as light sources 528 or simply light source 528 when referred to a single light source 528. In one example, light source 528 includes a germicidal lamp that produces or emits ultraviolet C (UVC) radiation with wavelengths between 200 and 290 nanometer (nm). When operated, light source 528 kills microorganisms by damaging the genetic material, and the deoxyribonucleic acid (DNA) of the microorganisms. In one example, light source 528 draws power from a power source (AC). In another example, light source 528 draws power using any other known methods, such as a battery, for example.

In accordance with one embodiment of the present invention, light fixtures 514 are fixed at predefined locations such that light sources 528 affixed to light fixtures 514 deliver an ultra-fast and effective dosage of UVC light for killing harmful pathogens when disinfecting closed space 516 or any particular area in closed space 616 such as chair 524, for example. As light fixtures 514 are fixed at the predefined locations, there is no need to move or position (or reposition) them with each use.

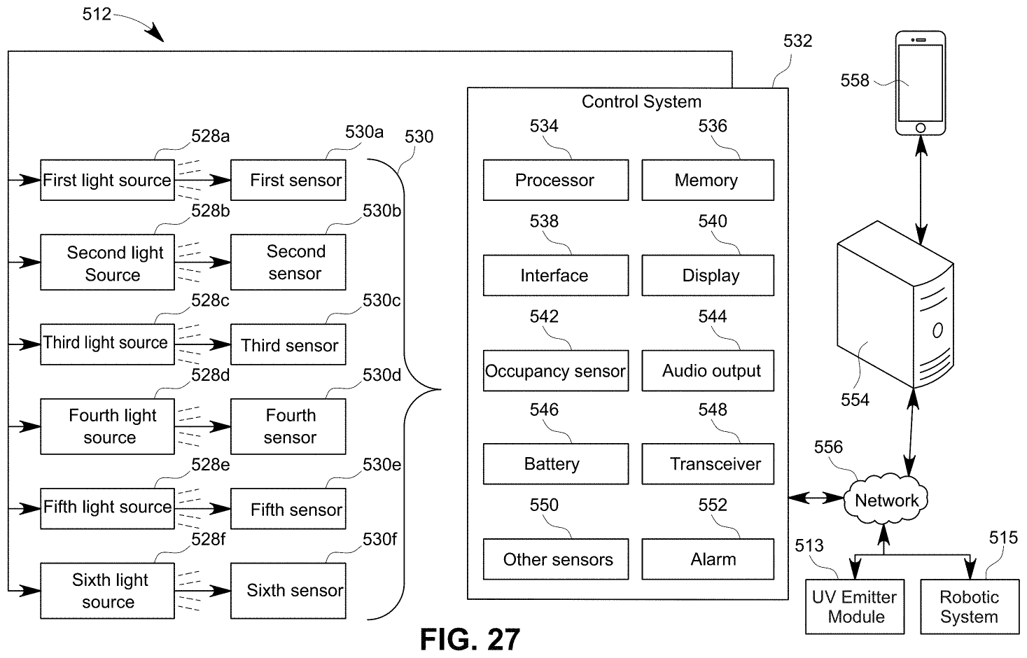

FIG. 27 shows a block diagram of room disinfection system 512, in accordance with one embodiment of the present invention. Room disinfection system 512 includes first light source 528a, second light source 528b, third light source 528c, fourth light source 528d, fifth light source 528e and sixth light source 528f that emit UVC light from their fixed (respective light fixture 514) locations. In accordance with one embodiment, room disinfection system 512 includes a first sensor 530a, a second sensor 530b, a third sensor 530c, a fourth sensor 530d, a fifth sensor 530e and a sixth sensor 530f, collectively referred as sensors 530 or simply sensor 530, when referred to a single sensor 530. Here, each sensor 530 mounts facing each light source 528. Alternatively, room disinfection system 512 includes only one sensor 530 that senses the amount of UVC light emitted or delivered from all light sources 528. In one implementation, each sensor 530 mounts at frame 526 of light fixture 514 and detects the amount of UVC light emitted from light source 528 present in that light fixture 514. In another implementation, sensor 530 mounts at chair 524 or any other place in closed space 516.

Sensors 530 sense UVC light delivered from each light source 528. In one example, each sensor 530 includes a UV sensor that detects ultraviolet light at high sensitivity. Sensors 530 measure ambient ultraviolet intensity in the 200 to 370 nm range. Further, sensors 530 log data corresponding to the ultraviolet intensity measured. After sensing, sensors 530 transmit the data to a control system 532 that processes the data and controls the operation of light sources 528. In one implementation, control system 532 mounts in closed space 516 as shown in FIG. 25B, for example. In another implementation, control system 532 installs outside closed space 516 or remotely from closed space 516 using wired or wireless systems.

Control system 532 includes a processor 534. Processor 534 includes one or more commonly known Central Processing Unit (CPU) such as a microprocessor or microcontroller. It should be understood that processor 534 is responsible for implementing specific functions under the control of software including an operating system, and any appropriate applications software.

Control system 532 includes a memory 536 such as a volatile memory (e.g., RAM), non-volatile memory (e.g., disk memory, FLASH memory, EPROMs, etc.), unalterable memory, and/or other types of memory. In one implementation, memory 536 stores data, program instructions. The program instructions might control the operation of an operating system and/or one or more applications.

Control system 532 includes interface(s) 538. Interface 538 includes wired interfaces and/or wireless interfaces. In at least one implementation, interface(s) 538 includes functionality similar to at least a portion of functionality implemented by one or more computer system interfaces such as those described herein and/or generally known to one having ordinary skill in the art.

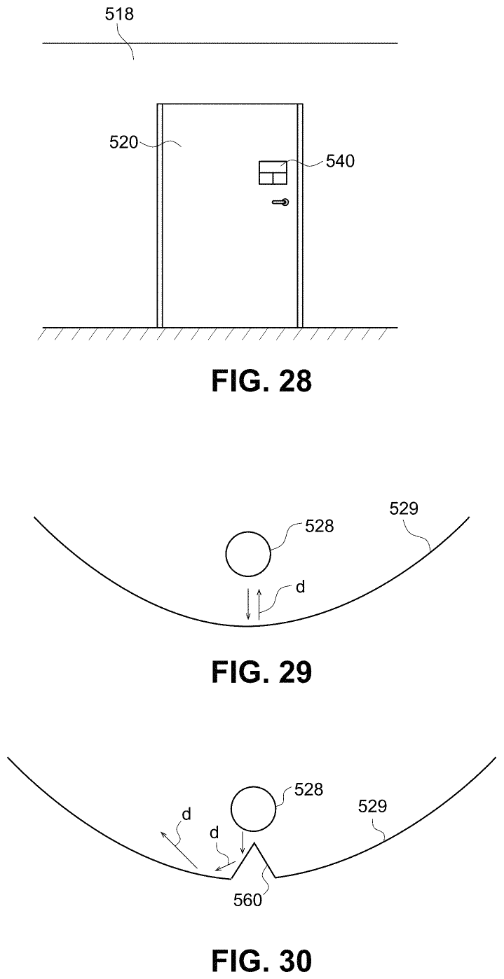

Control system 532 includes display(s) 40. Display(s) 40 uses LCD display technology, OLED display technology, and/or other types of conventional display technology. In one implementation, display 540 mounts at door 522 i.e., outside closed space 16. FIG. 28 shows display 540 installed at door 522 (outside of closed space 516), in accordance with one exemplary embodiment of the present invention. Display 40 mounts outside closed space 516 such that users (not shown) can view the status/operation (ON or OFF) of room disinfection system 512. In one implementation, display 540 includes a control panel (not shown) for controlling the operation (switching ON or OFF room disinfection system 512 (control system 532) or changing settings of room disinfection system 512 and/or light sources 528. In one example, the control panel has a capacitive touch capability for use as a “touch control panel” to control the operation of room disinfection system 512.

Control system 532 includes an occupancy sensor 542. Occupancy sensor 542 includes one of a camera, an infrared sensor, a motion sensor, and the like. Occupancy sensor 542 detects presence or movement of occupants in closed space 516. Occupancy sensor 542 senses presence of the occupants and transmits signals to processor 534 to turn OFF light sources 528. Upon obtaining signal, processor 534 turns OFF light sources 528 to prevent the occupants from getting exposed to harmful UVC light delivered by light sources 528.

Control system 532 includes an audio output 544. Audio output 544 indicates a speaker that receives the audio decoded by an Audio/Video unit(s) (not shown) and announces/provides voice instructions to the occupants in closed space 516.

Control system 532 includes a battery 546 configured to power control system 532 and/or light sources 528.

Control system 532 includes a wireless communication module/transceiver 548. Transceiver 548 configures to communicate with external devices such as a server 554 and/or an electronic device 558 using one or more wireless interfaces/protocols such as, for example, 802.11 (Wi-Fi), 802.15 (including Bluetooth™), 802.15 (Wi-Max), 802.22, Cellular standards such as CDMA, CDMA2000, WCDMA, Radio Frequency (e.g., RFID), Infrared, Near Field Magnetics, etc.

Control system 532 includes other sensors 550 such as proximity sensors, optical sensors, temperature sensors, and the like, or a combination thereof. Other sensors 550 work either as standalone sensors or in conjunction with occupancy sensor 542.

Control system 532 includes an alarm 552. Alarm 552 operates as a standalone speaker/buzzer or operates in conjunction with audio output 544 to generate an alert to the occupants inside or outside closed space 516 of the room disinfection system's operation (ON or OFF). In one example, alarm 552 produces sound when control system 532 completes a disinfection cycle in closed space 516 with the help of light sources 528 indicating to users or occupants that it is safe to enter closed space 516.

In accordance with the present invention, light fixtures 514 are fixed to walls 518 or ceiling 520 in such a way that UVC light emitted from light sources 528 are focused on chair 524 or any other point in closed space 516. In one implementation, reflector 529 is provided with a special geometric feature to minimize or eliminate UVC light emitted from light source 528 to be reflected back to light source 528. FIG. 29 shows reflector 529 having a curvature configuration, in accordance with one exemplary embodiment of the present invention. As can be seen, UVC light emitted from light source 528 is directed at a direction d such that UVC light gets reflected back to light source 528. This results in a decrease in the amount of UVC light delivered to chair 524 or any other point in closed space 516. In order to minimize and eliminate the UVC light getting reflected back to light source 528 and to enhance the utilization of the UVC light, reflector 529 is provided with a special geometric feature 560, as shown in FIG. 30. FIG. 30 shows geometric feature 560 in a triangular configuration raised from the curvature configuration whereby the sharp edge of the triangular configuration faces light source 528. In other words, the curvature configuration includes a bend-like structure to create a ridge (i.e., special geometric feature 560) that extends from the surface of reflector 529. The ridge eliminates the UVC light emitted from light source 528 from being reflected back into light source 528. However, a person skilled in the art understands geometric feature 560 can come in any other configuration to deflect or change direction d of the UVC light away from light source 528 after UVC light hits reflector 529. By changing direction d of the UVC light, geometric feature 560 minimizes or eliminates the UVC light emitted from light source 528 to be reflected back to light source 528. This increases the amount of the UVC light delivered to chair 524 or any other point in closed space 516.

When light sources 528 emit UVC light, sensors 530 capture the amount of UVC light delivered from light sources 528. Sensors 530 capture data such as amount of UVC delivered and the time period light sources 528 were active in delivering UVC light. Subsequently, sensors 530 transmit the data to control system 532. In one example, control system 532 operates light sources 528 for a predefined time period say two (2) minutes to deliver required UVC light to disinfect chair 524. In another example, control system 532 controls each of light sources 528 to deliver a certain amount of UVC light onto chair 524 to disinfect the surface of chair 524. As specified above, control system 532 operates light sources 528 to deliver the required amount of UVC dose depending on the size of closed space 516 or capacity of light sources 528 or time required to disinfect a certain surface. The fixed location of light fixtures 524 ensures light sources 528 deliver the UVC light at a specific location thereby reducing the time required to disinfect any surface in closed space 516 or entire closed space 516. In a hospital setting, this minimizes the time required to sanitize closed space 516 between each patient and allows using closed space 516 most of the time for providing medical care or treatment. Further, the location-specialized UVC dose is quickly delivered to target life-threatening, health care-associated infection (HAI) pathogens and disinfect an entire closed space 516 with higher efficacy.

In one embodiment, room disinfection system 512 determines the amount of UVC light (dose or dosage) to be delivered based on a profile of chair 524 or any specific point in closed space 516 or the entire closed space 516 (structure). For instance, room disinfection system 512 determines that entire closed space 516 requires more dose of UVC light for a period of time longer than chair 524. This is because; some closed spaces 516 have furniture, fixtures and equipment locations that do not change. Further, the size of closed space 516 may vary. Based on the profile of chair 524 or any specific point in closed space 516 or the entire closed space 516, control system 532 stores the data such as the amount of UVC light needed and corresponding time to sanitize.

When chair 524 or any specific point in closed space 516 or the entire closed space 516 needs to be sanitized, control system 532 looks up the profile and identifies the amount of UVC light to be delivered and corresponding time period to deliver the UVC light. For example, consider control system 532 has to sanitize the entire closed space 516, then control system 532 obtains the data such as the amount of UVC light to be delivered and corresponding time period to deliver the UVC light. Based on the data, control system 532 operates light sources 528 to deliver the UVC light for the predetermined time period. Similarly, the control system 532 obtains the data corresponding to the space to be sanitized and operates light sources 528 to deliver the UVC light for the predetermined time period. As control system 532 obtains the profile prior, it allows control system 532 to change the dose of the UVC light without having to recalibrate each time a dose is changed. Here, control system 532 utilizes the data from sensors 530 periodically and records the dose delivered to update the profile of the chair 524 or closed space 516 from time to time.

Optionally, control system 532 runs several calibration cycles with sensors, which are placed in different areas for each cycle. Upon completion of a few calibration cycles, control system 532 uses the profile for the cycle with the longest time, or a profile based upon a predetermined factor in order to deliver at least the target dosage to all areas where sensors 530 are placed for the calibration cycles.

At the time of delivering the UVC light, processor 534 instructs display 540 to display a message or instruction that disinfection is in process. After delivering the required amount of UVC light onto the surface of chair 524 or to closed space 516, control system 532 turns OFF light sources 528. Concurrently or consecutively, control system 532 instructs display 540 to display a message or instruction that disinfection is complete, and users are safe to enter closed space 516.

In one implementation, processor 534 employs occupancy sensor 542 to detect presence of occupants in closed space 516. Alternatively, processor 534 employs occupancy sensor 542 to check whether door 522 is closed. After detecting that no human is present in closed space 516 and/or door 522 is closed, then processor 534 operates light sources 528 for a predetermined time period as explained above. In one example, processor 534 automatically operates light sources 528 to deliver the required amount of UVC dose to chair 524 or in closed space 516 at predetermined time intervals, say once in an hour. At each hour, processor 534 employs occupancy sensor 542 to detect the presence of occupants in closed space 516 and/or checks whether door 522 is closed. Further, processor 534 operates light sources 528 to deliver the required amount of UVC light to closed space 516 or chair 524. If occupancy sensor 542 detects presence of occupants, then processor 534 employs audio output 544 to provide audio instructions or employs alarm 552 to sound an alert to occupants to exit closed space 516. Once occupants are out of closed space 516 and door 522 is shut, processor 534 operates light sources 528 for the predetermined time period to deliver the required amount of UVC light to closed space 516 or chair 524.

In one implementation, control system 532 communicatively connects to server 554 via a network 556. Server 554 includes a database that stores and processes a large amount of data. Network 556 includes a wireless network, a wired network or a combination thereof. Network 556 can be implemented as one of the different types of networks, such as intranet, local area network (LAN), wide area network (WAN), the internet, and the like. Network 556 implements as a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further the network 556 includes a variety of network devices, including routers, bridges, servers, computing devices, storage devices, and the like. Server 554 communicatively connects to electronic device 558 such as a mobile phone, tablet, desktop, smart watch and the like.

In the present implementation, control system 532 captures and/or transmits the data such as date, time, operator, room number, cycle time and delivered UVC dose to server 554. Assuming that room disinfection system 512 installs in closed space 516, control system 532 records the date, time, operator, room number, cycle time and delivered dose and transmits the information to server 554. Server 554 stores the data and utilizes the data for performing data analytics. A user of electronic device 558 accesses the data on server 554. In one implementation, the user of electronic device 558 operates control system 532 via server 554 to deliver required UVC dose through light sources 528, as explained above.

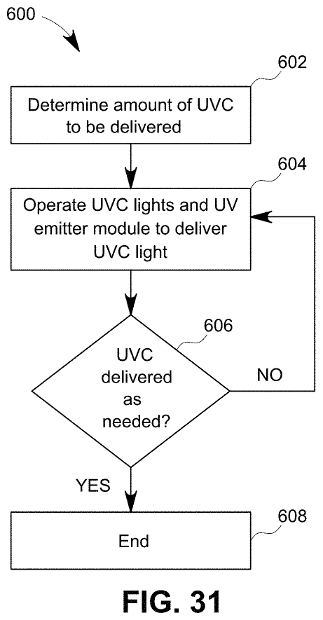

FIG. 31 shows a method 600 of disinfecting closed space or surface, in accordance with further embodiment of the present subject matter. Method 600 may be described in the general context of computer executable instructions. Generally, computer executable instructions may include routines, programs, objects, components, data structures, procedures, modules, functions, etc., that perform particular functions or implement particular abstract data types. Method 600 may also be practiced in a distributed computing environment where functions are performed by remote processing devices that are linked through a communications network. In a distributed computing environment, computer executable instructions may be located in both local and remote computer storage media, including memory storage devices.

The order in which method 600 is described and is not intended to be construed as a limitation, and any number of the described method blocks can be combined in any order to implement method 600 or alternate methods. Additionally, blocks may be deleted from method 600 without departing from the spirit and scope of the subject matter described herein. Furthermore, method 600 may be implemented in any suitable hardware, software, firmware, or combination thereof. However, for ease of explanation, in the embodiments described below, method 600 may be implemented in the above-described control system 532.

At step 602, control system 532 determines the amount of UVC light to be delivered by light sources 528 in closed space 516 or onto surfaces such as chair 524. As specified above, control system 532 operates light sources 528 at a predetermined time interval or when instructed by a user operating touch control system on display 540. Here, control system 532 operates UV emitter module 513 and light sources 528 to deliver (step 604) the required amount of UVC light for a predetermined time period, say 2 minutes. A person skilled in the art understands that control system 532 operates UV emitter module 513 and light sources 528 to deliver the required amount of UVC light depending on the size of closed space 516 or capacity of light sources 528 or time required to disinfect a certain surface. While UV emitter module 513 and light sources 528 are delivering the UVC light, control system 532 captures the amount of UVC light delivered using sensors 530 and checks whether the required amount of UVC light has been delivered (step 606). Once the required amount of the UVC light has been delivered, then control system 532 turns OFF UV emitter module 513 and light sources 528 and method 600 ends at step 608. If control system 532 determines that the required amount of UVC light has not been delivered, then method 600 moves back to step 604 where control system 532 continues to operate UV emitter module 513 and light sources 528 until the required amount of UVC light is delivered.

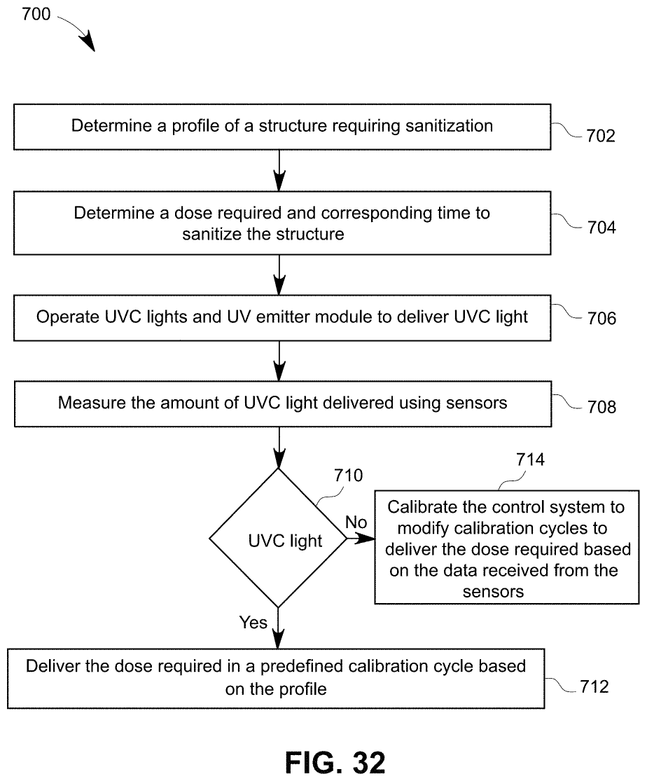

FIG. 32 shows a method 700 of disinfecting a closed space or surface by calibrating a control system based on the profile of the closed space or surface, in accordance with further embodiment of the present subject matter. Method 700 may be described in the general context of computer executable instructions. Generally, computer executable instructions may include routines, programs, objects, components, data structures, procedures, modules, functions, etc., that perform particular functions or implement particular abstract data types. Method 700 may also be practiced in a distributed computing environment where functions are performed by remote processing devices that are linked through a communications network. In a distributed computing environment, computer executable instructions may be located in both local and remote computer storage media, including memory storage devices.

The order in which method 700 is described and is not intended to be construed as a limitation, and any number of the described method blocks can be combined in any order to implement method 700 or alternate methods. Additionally, blocks may be deleted from method 700 without departing from the spirit and scope of the subject matter described herein. Furthermore, method 700 may be implemented in any suitable hardware, software, firmware, or combination thereof. However, for ease of explanation, in the embodiments described below, method 700 may be implemented in the above-described control system 532.

Method 700 starts at step 702. At step 702, control system 532 determines a profile of a structure that requires sanitization. An example of structure includes, but not limited to, furniture, fixtures, equipment, or entire closed space (room), etc. Based on the structure, control system 532 determines a dose i.e., amount of UVC needed and corresponding time to sanitize the structure, as shown at step 704. At step 706, control system 532 operates UV emitter module 513 and light sources 528 to deliver UVC light. Concurrently or consecutively, control system 532 obtains/monitors the amount of UVC light delivered by UV emitter module 513 and light sources 528 from sensors 530, as shown at step 708.

At step 710, control system 532 checks whether UVC light is delivered as needed based on the profile. If the required dose is delivered, then method 700 moves to step 712. At step 712, control system 732 operates UV emitter module 513 and light sources 528 to deliver the dose required in a predefined calibration cycle based on the profile. If control system 532 determines that UVC light is not delivered as needed at step 710, then method 700 moves to step 714. At step 714, control system 532 gets calibrated to modify the calibration cycles to deliver the dose based on the data received from sensors 530. This allows control system 532 to change dose of the UVC light without having to recalibrate each time a dose is changed.

Based on the above, it is evident that the room disinfection system provides an ultra-fast and effective delivery of UVC light for killing harmful pathogens and disinfecting closed spaces (rooms), or any other surface without having to be moved or positioned with each use.

The present invention has been described in particular detail with respect to various possible embodiments, and those of skill in the art will appreciate that the invention may be practiced in other embodiments. First, the particular naming of the components, capitalization of terms, the attributes, data structures, or any other programming or structural aspect is not mandatory or significant, and the mechanisms that implement the invention or its features may have different names, formats, or protocols. Further, the system may be implemented via a combination of hardware and software, as described, or entirely in hardware elements. In addition, the particular division of functionality between the various system components described herein is merely exemplary, and not mandatory; functions performed by a single system component may instead be performed by multiple components, and functions performed by multiple components may instead performed by a single component.

Some portions of above description present the features of the present invention in terms of algorithms and symbolic representations of operations on information. These algorithmic descriptions and representations are the means used by those skilled in the data processing arts to most effectively convey the substance of their work to others skilled in the art. These operations, while described functionally or logically, should be understood as being implemented by computer programs.

Further, certain aspects of the present invention include process steps and instructions described herein in the form of an algorithm. It should be noted that the process steps and instructions of the present invention could be embodied in software, firmware or hardware, and when embodied in software, could be downloaded to reside on and be operated from different platforms used by real time network operating systems.

The algorithms and operations presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will be apparent to those of skill in the, along with equivalent variations. In addition, the present invention is not described with reference to any particular programming language. It is appreciated that a variety of programming languages may be used to implement the teachings of the present invention as described herein, and any references to specific languages are provided for disclosure of enablement and best mode of the present invention.

The foregoing description of the preferred embodiments is provided to enable persons skilled in the art to make and use the present invention. Various modifications to these embodiments will be readily apparent to the persons skilled in the art. The scope of the present invention is limited only by the appended claims, and their legal equivalents.

What is claimed is:

1. A disinfection system for disinfecting a closed space or surface, said disinfection system comprising:
a plurality of light fixtures fixedly positioned at predefined locations in a closed space, wherein each of said plurality of light fixtures comprises a light source, wherein said light source emits ultraviolet C (UVC) light for disinfecting said closed space or a surface in said closed space;
a robotic system;
a UV emitter module positioned over said robotic system, wherein said UV emitter module comprises an Ultraviolet (UV) light source for disinfecting said closed space or said surface in said closed space;
a plurality of sensors for measuring the amount of UVC light delivered by said light sources and said UV emitter module; and
a control system for determining a profile for said closed space or said surface based on the amount of UVC light needed and corresponding time to disinfect said closed space or said surface,
wherein said control system communicatively connects said plurality of sensors, and
wherein said control system monitors the amount of UVC light delivered by said light sources and said UV emitter module using said plurality of sensors and controls the operation of said light sources and said UV emitter module to deliver the required amount of the UVC light based on the profile for said closed space or said surface.

2. The disinfection system of claim 1, wherein each of said plurality of light fixtures comprises a reflector, wherein said reflector comprises a special geometric feature, and wherein said reflector maximizes UVC light produced by said light source and minimizes the time required to disinfect said closed space or said surface in said closed space.

3. The disinfection system of claim 1, wherein said plurality of light fixtures mounts at walls or ceiling of the closed space, and wherein said plurality of light fixtures are aligned such that said light sources deliver the UVC light at a specific point in said closed space.

4. The disinfection system of claim 1, wherein said special geometric feature comprises a curvature configuration, wherein said curvature configuration comprises a ridge extending from said curvature configuration facing said light source, and wherein said ridge minimizes or eliminates the UVC light emitted from said light source to be reflected back to said light source.

5. The disinfection system of claim 1, wherein said control system turns OFF said light sources and said UV emitter module upon delivery of required amount of UVC light.

6. The disinfection system of claim 1, wherein said plurality of sensors captures time, number of cycles and amount of the UVC delivered by said light sources and said UV emitter module.

7. The disinfection system of claim 1, wherein said control system comprises a display configured for displaying a status of operation of said light sources and said UV emitter module.

8. The disinfection system of claim 1, wherein said control system comprises a display having a touch control panel, and wherein said touch control panel configures to operate the control system.

9. The disinfection system of claim 1, wherein said control system transmits information received from said sensors to a server, and wherein said server performs data analytics on the information.

10. The disinfection system of claim 1, wherein said robotic system receives a signal corresponding to discharge of a battery placed with said UV emitter module, wherein said robotic system detects location of said UV emitter module and maneuvers said UV emitter module to a charging dock for charging said battery, and wherein said robotic system maneuvers said UV emitter module to a predefined area for disinfecting said predefined area upon charging.

11. The disinfection system of claim 1, further comprises occupancy sensors configured to detect presence of humans in said closed space, wherein said occupancy sensors signal said control system to turn OFF said light sources and said UV emitter module upon detecting the presence of the humans.

12. The disinfection system of claim 1, wherein said robotic system comprises occupancy sensors configured to detect presence of humans in said closed space or along route of said robotic system while maneuvering, wherein robotic system terminates maneuvering upon detecting the presence of humans in said closed space or along the route while maneuvering.

13. The disinfection system of claim 1, wherein said robotic system comprises an emitter interface for mounting said UV emitter module.

14. A method of disinfecting a closed space or surface, said method comprising the steps of:
providing a plurality of light fixtures fixedly positioned at predefined locations in a closed space, each of said plurality of light fixtures comprising a light source, said light source emitting ultraviolet C (UVC) light for disinfecting said closed space or a surface in said closed space;
providing a robotic system;
providing a UV emitter module over said robotic system, said UV emitter module comprising an Ultraviolet (UV) light source for disinfecting said closed space or said surface in said closed space;
providing a plurality of sensors for measuring the amount of UVC light delivered by said light sources and said UV emitter module;
providing a control system for determining a profile for said closed space or said surface based on the amount of UVC light needed and corresponding time to disinfect said closed space or said surface;
communicatively connecting said control system and said plurality of sensors;
monitoring the amount of UVC light delivered by said light sources and said UV emitter module using said plurality of sensors; and
controlling the operation of said light sources and said UV emitter module to deliver the required amount of the UVC light based on the profile for said closed space or said surface.

15. The method of claim 14, further comprising providing a reflector at said a light fixture of said plurality of light fixtures, said reflector comprising a special geometric feature, said reflector maximizing UVC light produced by said light source and minimizing the time required to disinfect said closed space or said surface in said closed space.

16. The method of claim 14, further comprising turning OFF said light sources and said UV emitter module upon delivery of required amount of UVC light.

17. The method of claim 14, further comprising transmitting, by said control system, information received from said sensors to a server for performing data analytics on the information.

18. The method of claim 14, further comprising:
receiving, by said robotic system, a signal corresponding to discharge of a battery placed within said UV emitter module;
detecting, by said robotic system, location of said UV emitter module;
maneuvering, by said robotic system, said UV emitter module to a charging dock for charging said battery; and
maneuvering, by said robotic system, said UV emitter to a predefined area for disinfecting said predefined area upon charging.

19. The method of claim 14, further comprising:
providing occupancy sensors for detecting presence of humans in said closed space; and
turning off said light sources and said UV emitter module when said occupancy sensors signal detect the presence of the humans.

20. The method of claim 14, further comprising providing an emitter interface at said robotic system for mounting said UV emitter module.
